(19) United States
(12) Patent Application Publication
(10) Pub. No.: US 2023/0200210 A1
(43) Pub. Date: Jun. 22, 2023

(54) ONCN QUADRIDENTATE LIGAND-CONTAINING PLATINUM COMPLEX AND APPLICATION THEREOF IN ORGANIC LIGHT-EMITTING DIODE

(71) Applicant: GUANGDONG AGLAIA OPTOELECTRONIC MATERIALS CO., LTD, Foshan, Guangdong (CN)

(72) Inventors: Huiyang LI, Foshan (CN); Lei DAI, Foshan (CN); Lifei CAI, Foshan (CN)

(73) Assignee: GUANGDONG AGLAIA OPTOELECTRONIC MATERIALS CO., LTD, Foshan, Guangdong (CN)

(21) Appl. No.: 17/926,969

(22) PCT Filed: May 9, 2021
(86) PCT No.: PCT/CN2021/092523

§ 371 Date: Nov. 21, 2022

(30) Foreign Application Priority Data
May 26, 2020 (CN) 202010456878.1

(51) Int. Cl. H10K 85/30; C07F 15/00; C07D 401/14; C07D 471/04; C07D 487/04; C07D 495/04; C07D 491/048; C09K 11/06

(52) U.S. Cl. CPC H10K 85/346; C07F 15/0086; C07D 401/14; C07D 471/04; C07D 487/04; C07D 495/04; C07D 491/048; C09K 11/06; C09K 2211/185; C09K 2211/1044; H10K 50/11

(57) ABSTRACT

The present invention relates to an ONCN quadridentate ligand-containing platinum complex and application thereof in an organic light-emitting diode. The platinum complex is a compound having the structure as shown in a chemical formula (I). When the compound is applied to an organic light-emitting diode, low driving voltage and high luminescence efficiency are achieved, and the service life of a device can be significantly prolonged. The complex has a potential to be applied in the field of organic electroluminescent devices. The present invention further provides an organic electroluminescent device. The device includes a cathode, an anode, and an organic layer. The organic layer is one or more of a hole injection layer, a hole transport layer, a light-emitting layer, a hole blocking layer, an electron transport layer, and an electron injection layer, in which at least one of the layers includes the compound as shown in the structural formula (I).

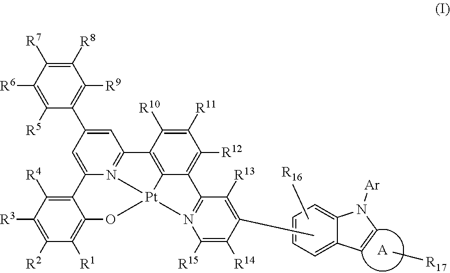

### TECHNICAL FIELD

The present invention relates to the field of luminescent materials, and specifically relates to an ONCN quadridentate ligand-containing platinum complex and application thereof in an organic light-emitting diode.

### BACKGROUND

Organic photoelectronic devices include, but are not limited to the following types: organic light-emitting diodes (OLEDs), organic thin-film transistors (OTFTs), organic photovoltaic devices (OPVs), light-emitting electrochemical cells (LCEs), and chemical sensors.

In recent years, as a lighting and display technology with a great application prospect, the OLEDs have attracted wide attention from the academia and industry. Due to the characteristics of self-lighting, wide viewing angle, short reaction time and the ability to prepare flexible devices, OLEDs devices have become a strong competitor in the lighting and display technology in the next generation. However, due to low efficiency, short service life and other problems, the current OLEDs need to be further studied.

According to early fluorescent OLEDs, only singlet luminescence can be generally used, and triplet excitons generated in the devices cannot be effectively used and are returned to the ground state by a non-radiative way, so that the promotion and use of the OLEDs are limited. An electrophosphorescence phenomenon was reported by Che Chi-Ming et al. from the University of Hong Kong for the first time in 1998. In the same year, phosphorescent OLEDs were prepared by Thompson et al. with a transition metal complex as a luminescent material. According to the phosphorescent OLEDs, singlet and triplet excitons can be efficiently used for luminescence, and an internal quantum efficiency of 100% can be achieved theoretically, so that the commercialization of the OLEDs is promoted to a large extent. The light-emitting color of the OLEDs can be controlled by structural design of luminescent materials. The OLEDs may include one or more of light-emitting layers to achieve a desired spectrum. At present, the commercialization of green, yellow, and red phosphorescent materials has been achieved. In commercial OLEDs displays, a combination of a blue fluorescence, a yellow or green phosphorescence, and a red phosphorescence is generally used to achieve full-color display. At present, luminescent materials with higher efficiency and longer service life are in an urgent need in the industry. Metal complex luminescent materials have been applied in the industry. However, properties, such as luminescence efficiency and service life, still need to be further improved.

### SUMMARY

In view of the above problems of the prior art, the present invention provides an ONCN quadridentate ligand-containing platinum complex luminescent material. When the material is applied in an organic light-emitting diode, great photoelectric properties and device service life are achieved.

The present invention further provides an organic light-emitting diode based on the platinum complex.

The ONCN quadridentate ligand-containing platinum complex is a compound having the structure as shown in a formula (I):

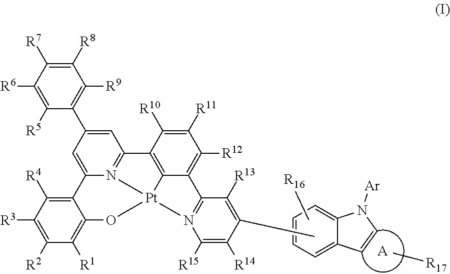

where

each of R¹ to R¹⁷ is independently selected from hydrogen, deuterium, halogen, amino, carbonyl, carboxyl, thioalkyl, cyano, sulfonyl, phosphino, substituted or unsubstituted alkyl containing 1-20 carbon atoms, substituted or unsubstituted cycloalkyl containing 3-20 carbon atoms, substituted or unsubstituted alkenyl containing 2-20 carbon atoms, substituted or unsubstituted alkoxyl containing 1-20 carbon atoms, substituted or unsubstituted aryl containing 6-30 carbon atoms, and substituted or unsubstituted heteroaryl containing 3-30 carbon atoms; or any two adjacent substituents are connected or condensed into a ring;

Ar is selected from substituted or unsubstituted aryl containing 6-30 carbon atoms and substituted or unsubstituted heteroaryl containing 3-30 carbon atoms;

A is a five-membered or six-membered aromatic ring or heteraromatic ring;

a heteroatom in the heteroaryl or heteroaromatic ring includes one or more of N, S, and O;

and the “substituted” refers to substitution with halogen, amino, cyano, or C₁-C₄ alkyls.

Preferably, each of the R¹ to R¹⁷ is independently selected from hydrogen, deuterium, halogen, amino, thioalkyl, cyano, substituted or unsubstituted alkyl containing 1-6 carbon atoms, substituted or unsubstituted cycloalkyl containing 3-6 carbon atoms, substituted or unsubstituted alkenyl containing 2-6 carbon atoms, substituted or unsubstituted alkoxyl containing 1-6 carbon atoms, substituted or unsubstituted aryl containing 6-12 carbon atoms, or substituted or unsubstituted heteroaryl containing 3-6 carbon atoms;

and the Ar is selected from substituted or unsubstituted aryl containing 6-12 carbon atoms and substituted or unsubstituted heteroaryl containing 3-12 carbon atoms.

Preferably, each of the R¹ to R¹⁷ is independently selected from hydrogen, deuterium, halogen, C₁-C₄ alkyls, cyano, substituted or unsubstituted cycloalkyl containing 3-6 carbon atoms, substituted or unsubstituted aryl containing 6-12 carbon atoms, and substituted or unsubstituted heteroaryl containing 3-6 carbon atoms;

and the Ar is selected from substituted or unsubstituted aryl containing 6-12 carbon atoms and substituted or unsubstituted heteroaryl containing 3-12 carbon atoms.

Preferably, each of the R¹ to R¹⁷ is independently selected from hydrogen, deuterium, methyl, isopropyl, isobutyl, tert-butyl, cyano, substituted or unsubstituted cyclopentyl, substituted or unsubstituted cyclohexyl, substituted or unsubstituted phenyl, substituted or unsubstituted pyridyl, substituted or unsubstituted pyrazinyl, and substituted or unsubstituted pyrimidinyl;

the Ar is selected from substituted or unsubstituted phenyl, substituted or unsubstituted pyridyl, substituted or unsubstituted pyrazinyl, and substituted or unsubstituted pyrimidinyl;

the A is selected from a benzene ring, a pyridine ring, a pyrazine ring, a pyrimidine ring, a thiophene ring, a furan ring, a pyrazole ring, and an imidazole ring;

and the “substituted” refers to substitution with halogen, cyano, or C₁-C₄ alkyls.

Preferably, in the general formula (I), each of the R¹ to R¹⁷ is independently selected from hydrogen, deuterium, methyl, tert-butyl, cyano, substituted or unsubstituted cyclopentyl, substituted or unsubstituted cyclohexyl, or substituted or unsubstituted phenyl;

the Ar is selected from substituted or unsubstituted phenyl and substituted or unsubstituted pyridyl;

and the A is selected from a benzene ring, a pyridine ring, a pyrazine ring, and a pyrimidine ring.

Further preferably, in the general formula (I), each of the R¹ to R¹⁷ is independently selected from hydrogen, deuterium, and tert-butyl;

the Ar is selected from phenyl, cyanophenyl, and pyridyl;

and the A is selected from a benzene ring, a pyridine ring, a pyrazine ring, and a pyrimidine ring.

Further preferably, in the general formula (I), among the R¹ to R¹⁷, R⁶ and R⁸ are tert-butyl, and the other groups are hydrogen;

the Ar is selected from phenyl and cyanophenyl;

and the A is selected from a benzene ring and a pyridine ring.

Examples of the platinum metal complex of the present invention are listed below. However, the complex is not limited to the structures listed.

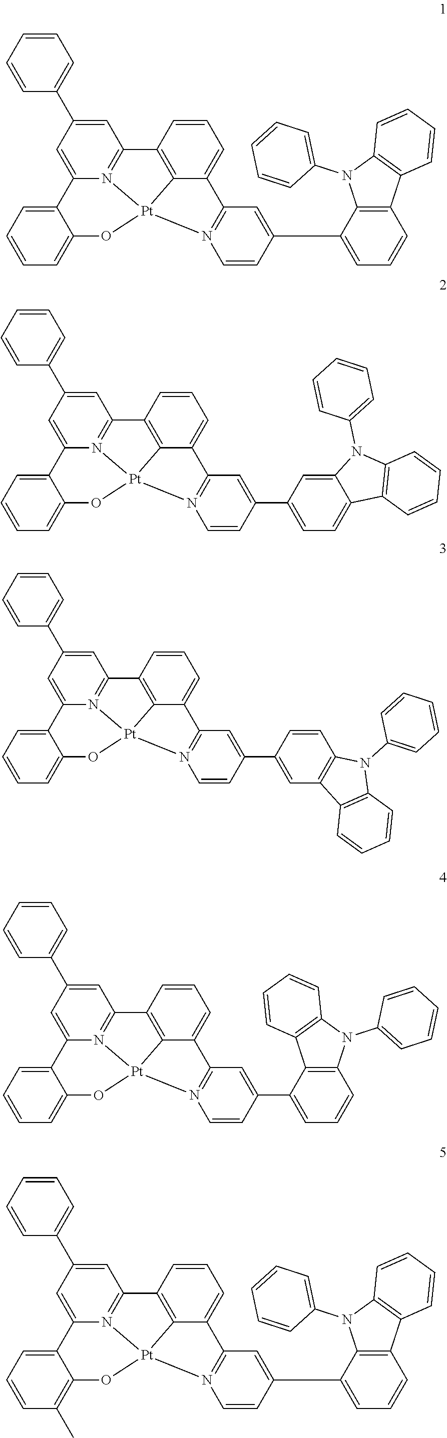

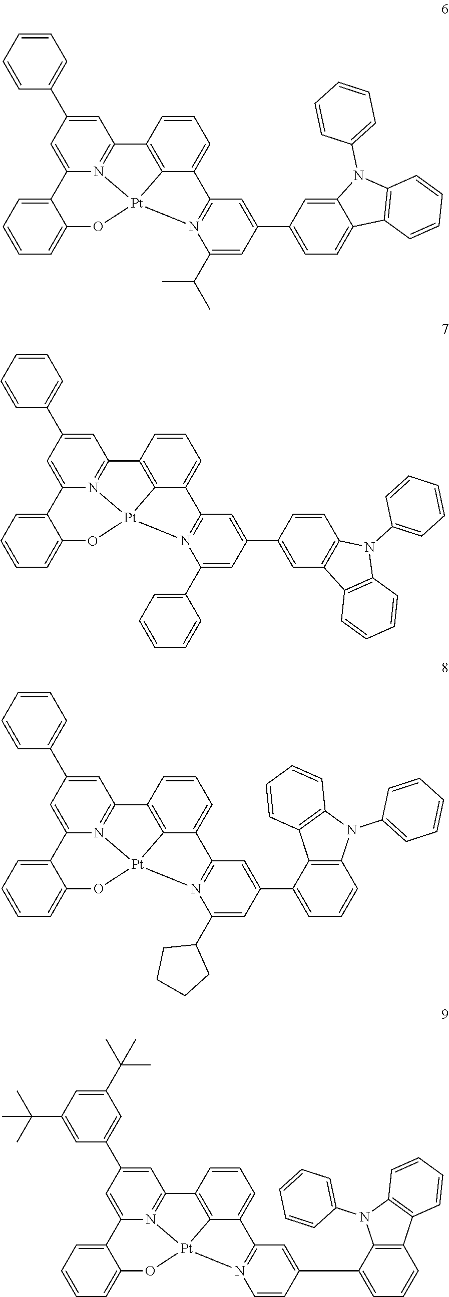

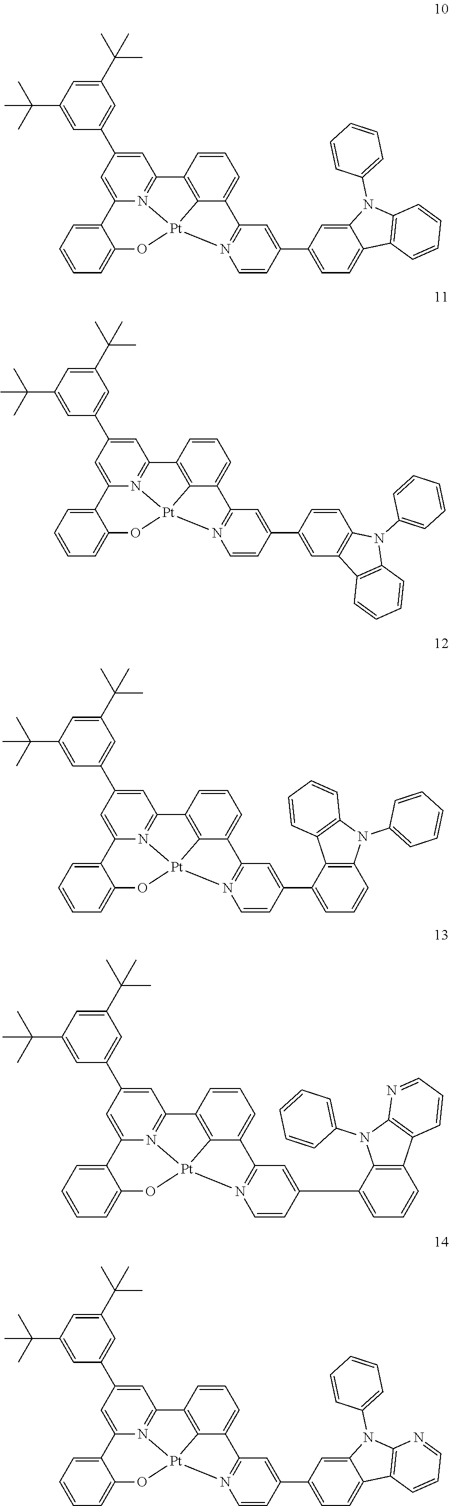

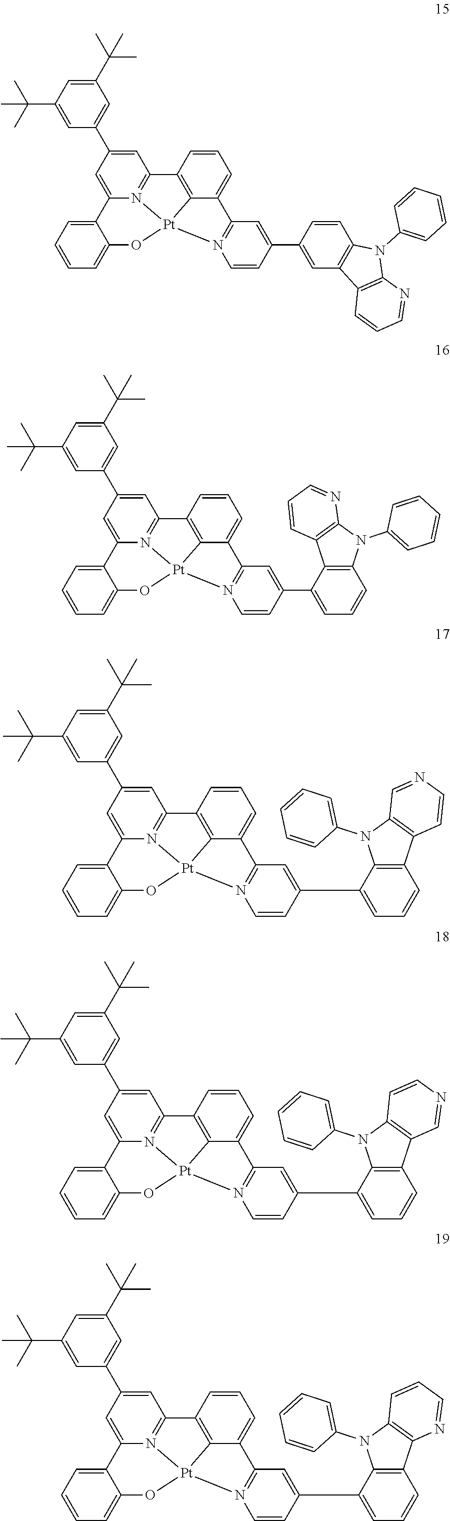

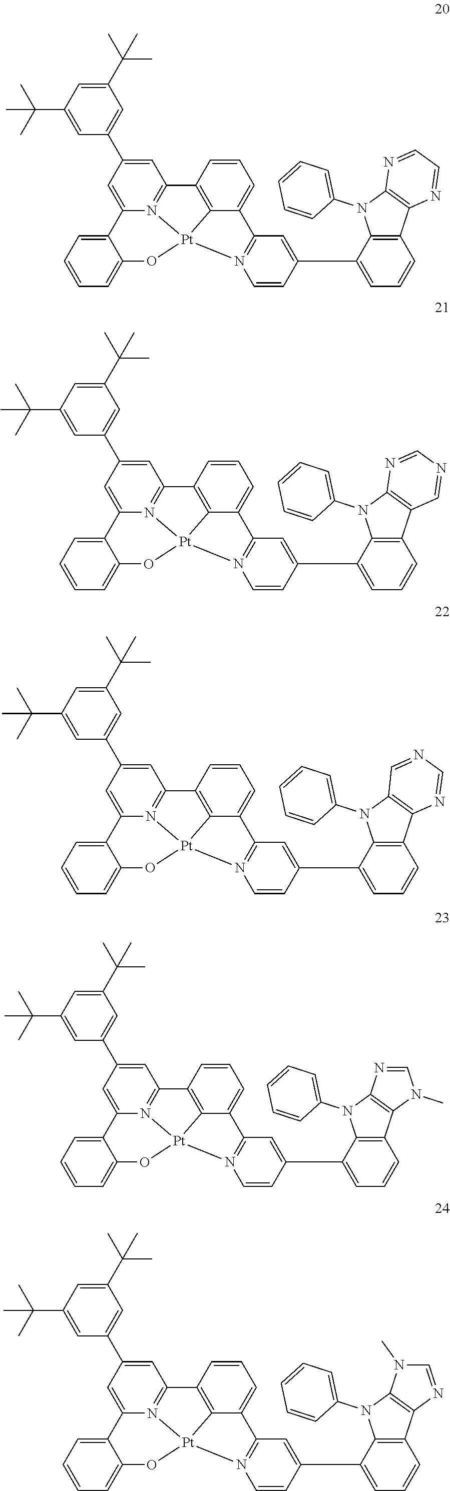

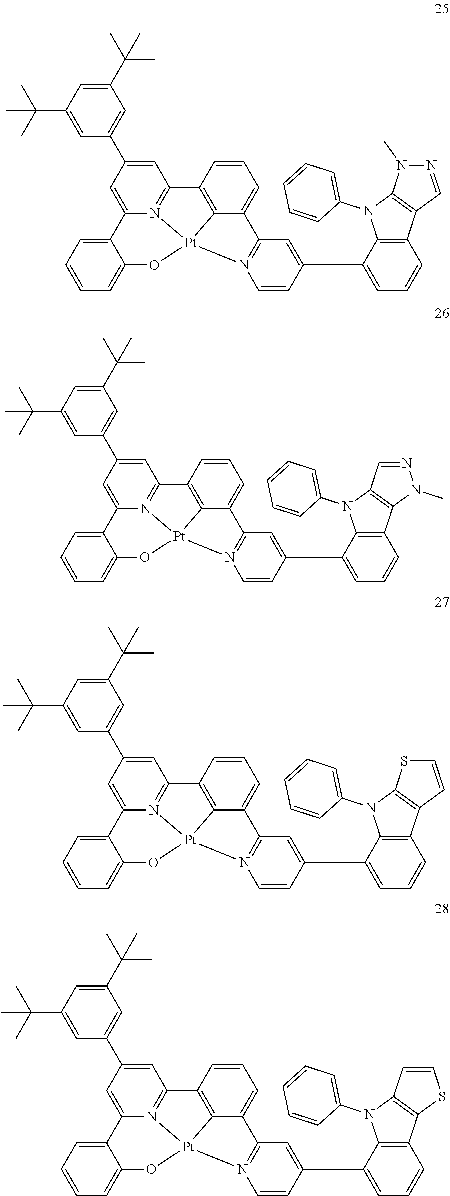

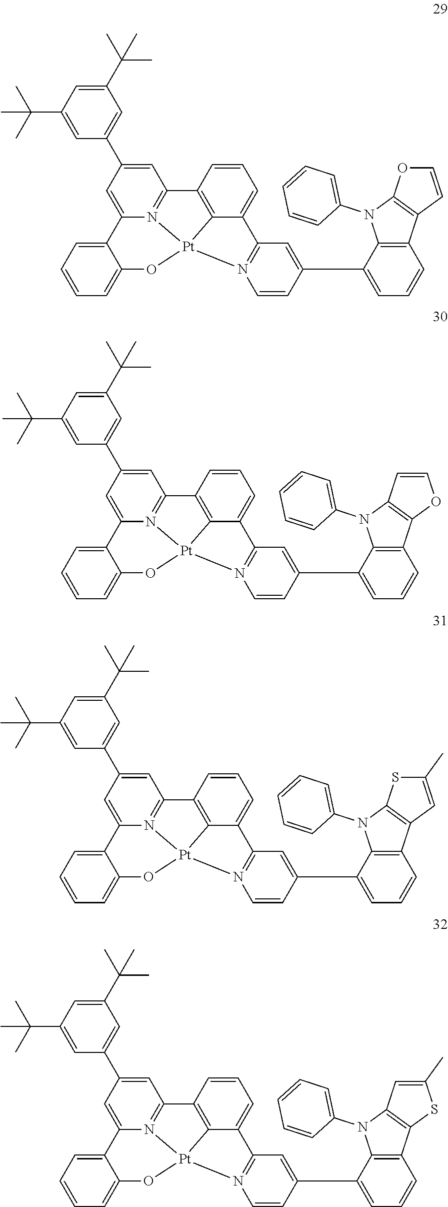

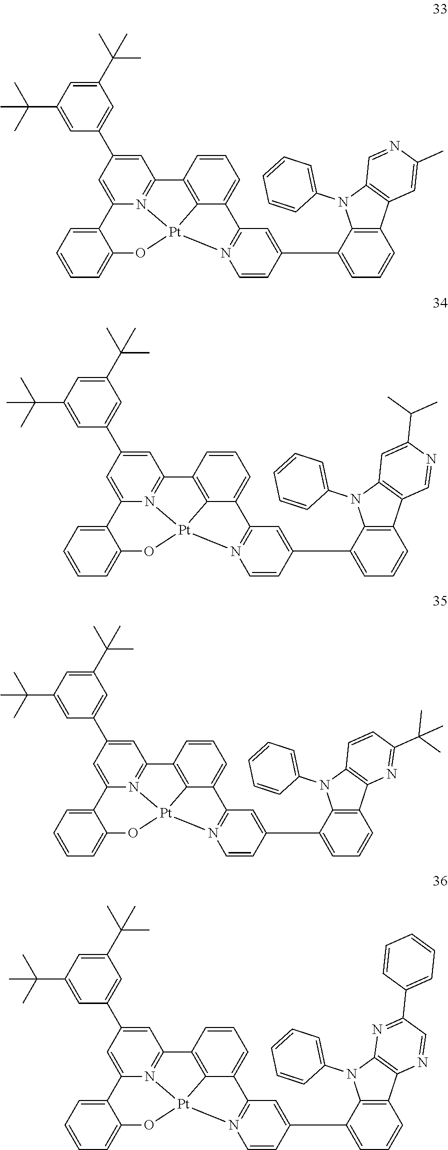

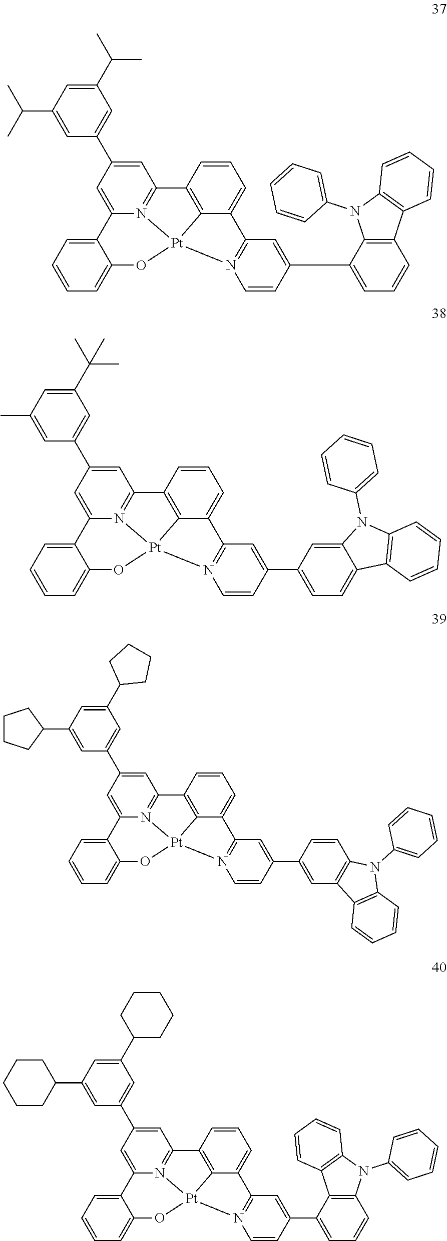

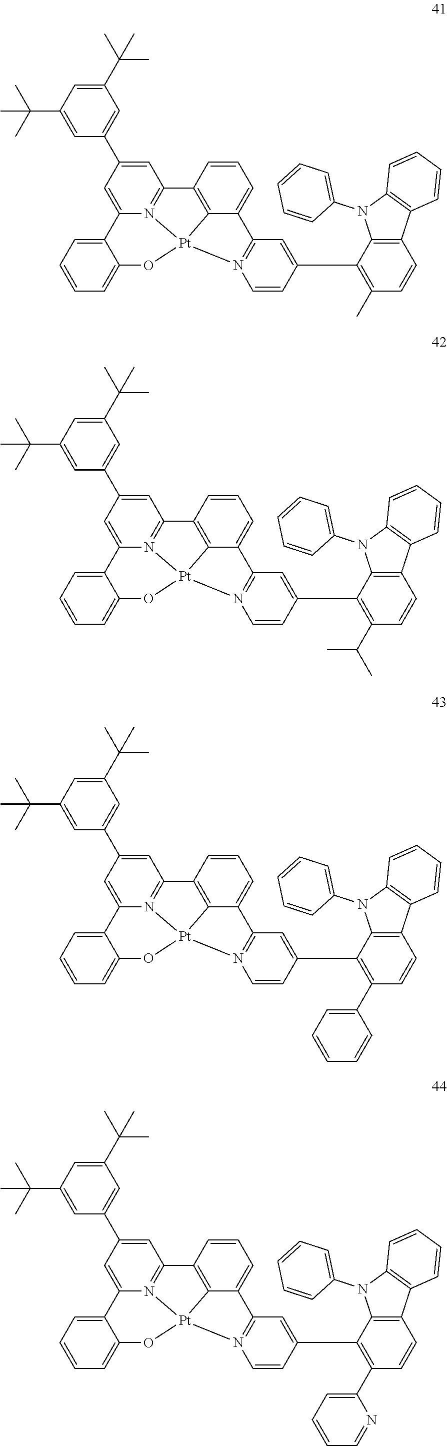

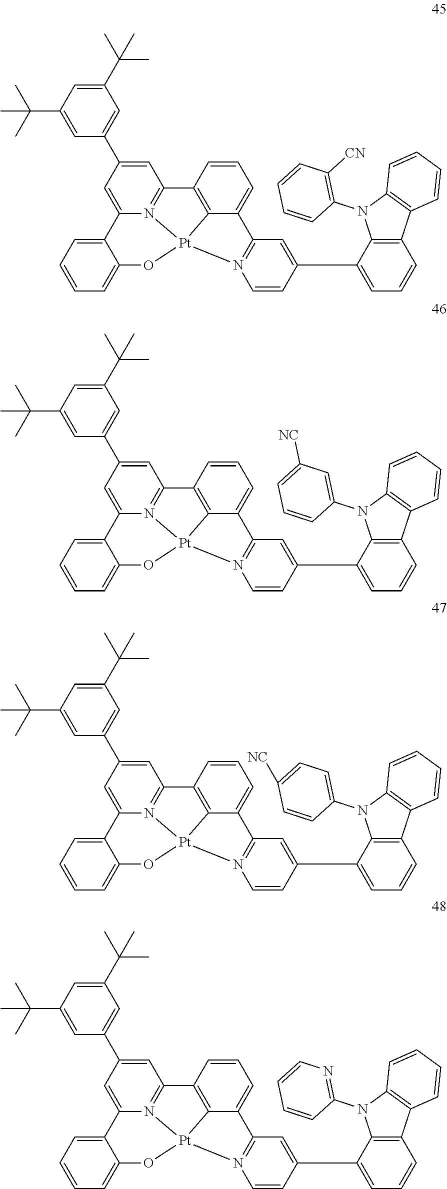

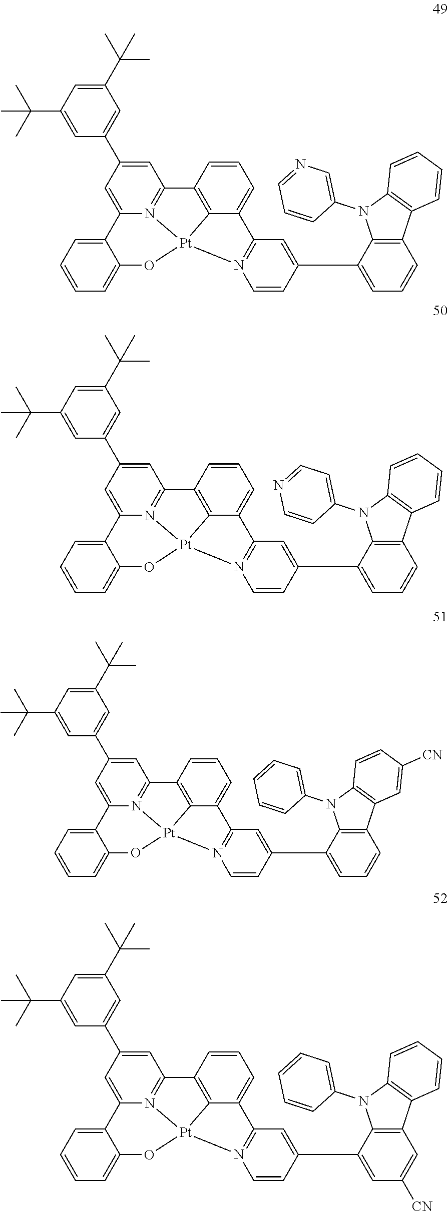

A precursor, that is ligand, of the metal complex has the following structural formula:

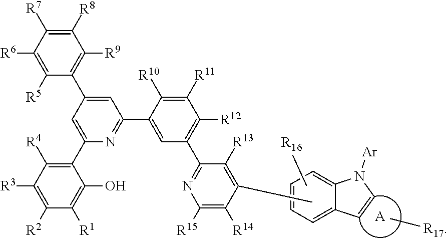

The present invention further provides application of the platinum complex in organic photoelectronic devices. The photoelectronic devices include, but are not limited to, organic light-emitting diodes (OLEDs), organic thin-film transistors (OTFTs), organic photovoltaic devices (OPVs), light-emitting electrochemical cells (LCEs), and chemical sensors, preferably OLEDs.

An organic light-emitting diode (OLEDs) includes the platinum complex, which is used as a luminescent material in a light-emitting device.

The organic light-emitting diode of the present invention includes a cathode, an anode, and an organic layer. The organic layer is one or more of a hole injection layer, a hole transport layer, a light-emitting layer, a hole blocking layer, an electron injection layer, and an electron transport layer, all of which are not required to exist at one time. At least one of the hole injection layer, the hole transport layer, the hole blocking layer, the electron injection layer, the light-emitting layer, and the electron transport layer includes the platinum complex as shown in the formula (I).

Preferably, a layer where the platinum complex as shown in the formula (I) is located is the light-emitting layer or the electron transport layer.

The organic layer of the device in the present invention has a total thickness of 1-1,000 nm, preferably 1-500 nm, and more preferably 5-300 nm.

The organic layer can be formed into a thin film by an evaporation or solution method.

A series of platinum complex luminescent materials with novel structures disclosed in the present invention have unexpected features, so that the luminescent efficiency and device service life of such compounds are significantly improved. Moreover, great thermal stability is achieved, so that requirements of OLED panels for luminescent materials are met.

### BRIEF DESCRIPTION OF DRAWINGS

FIG. 1 a structural diagram of an organic light-emitting diode device of the present invention.

In the figure, 10 refers to a glass substrate, 20 refers to an anode, 30 refers to a hole injection layer, 40 refers to a hole transport layer, 50 refers to a light-emitting layer, 60 refers to an electron transport layer, 70 refers to an electron injection layer, and 80 refers to a cathode.

### DETAILED DESCRIPTION OF EMBODIMENTS

Methods for synthesizing materials are not required in the present invention. In order to describe the present invention in more detail, the following examples are provided, but the present invention is not limited thereto. Unless otherwise specified, the following raw materials used during synthesis are commercially available products.

### EXAMPLE 1

Synthesis of a Complex 9

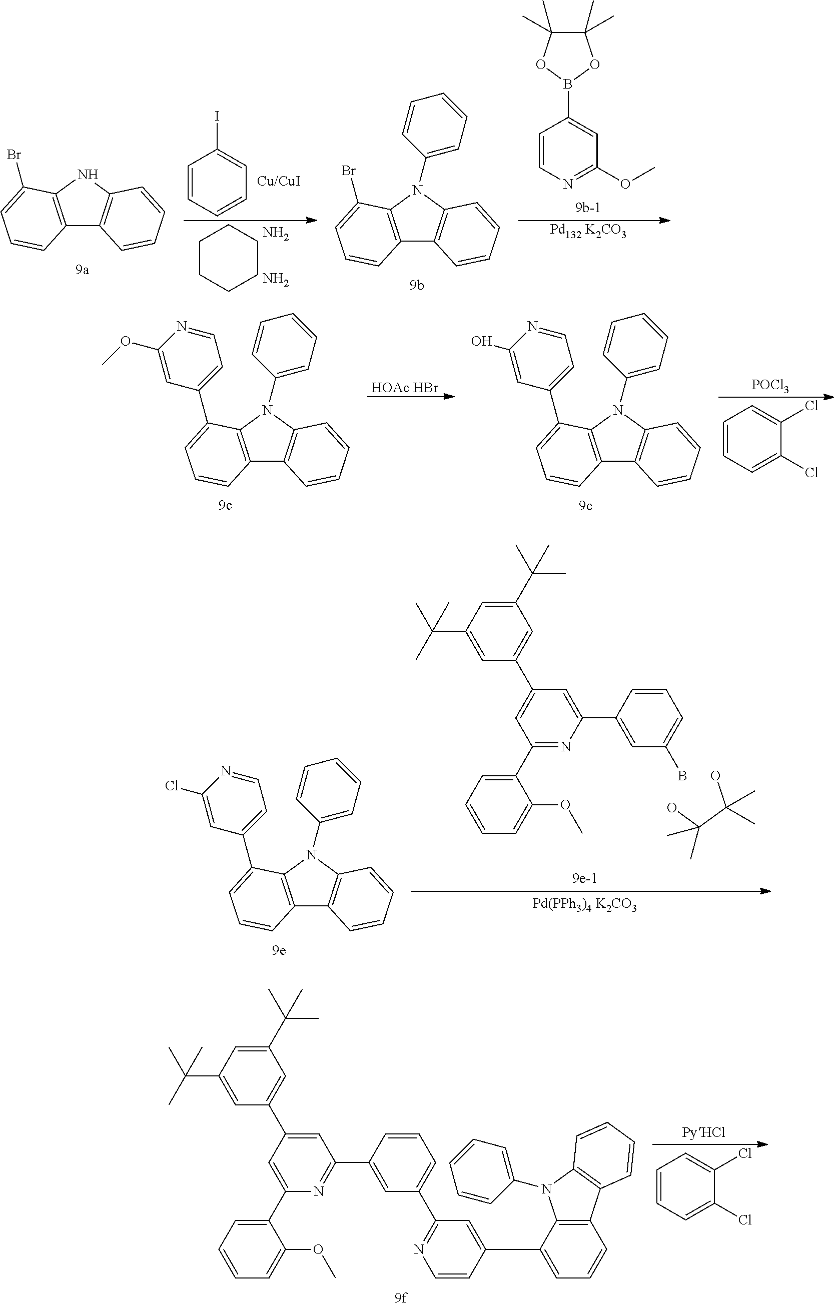

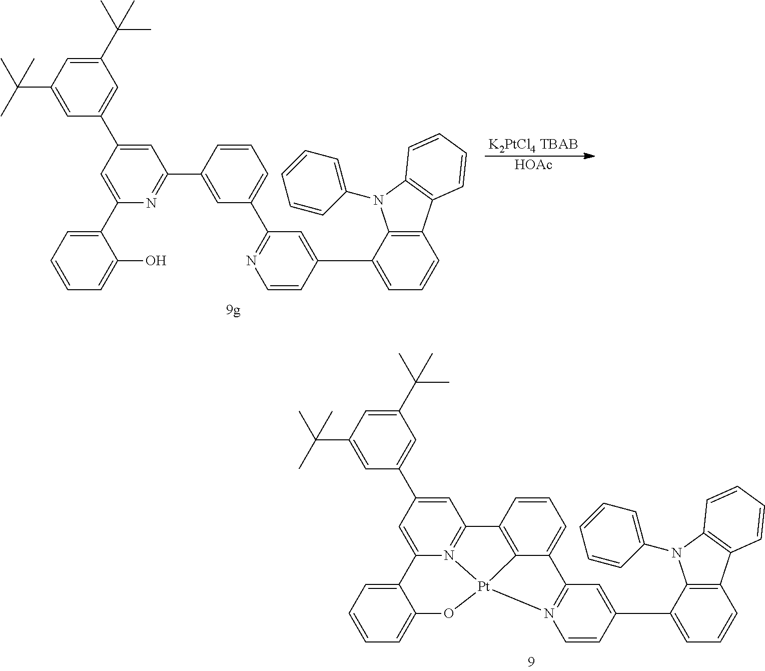

Synthesis of a Compound 9b

1-bromocarbazole (20 g, 81.2 mmol), iodobenzene (32 g, 162.4 mmol), cuprous iodide (1.4 g, 8.12 mmol), a copper powder (0.44 g, 8.12 mmol), 1,2-cyclohexenediamine (1.8 g, 16.24 mmol), and xylene (150 ml) were added to a flask, and stirred for a reaction at 100° C. for 12 hours under the protection of nitrogen. After the reaction was completed, toluene (100 ml) was used for rinsing twice. A solvent was spun out, and a residue was separated by column chromatography to obtain 6.2 g of a colorless oily product with a yield of 23.7%. ¹H NMR (400 MHz, Chloroform-d) δ 8.11 (d, J=7.7 Hz, 2H), 7.59-7.51 (m, 4H), 7.44 (dd, J=7.1, 2.3 Hz, 2H), 7.38 (d, J=8.2 Hz, 1H), 7.31 (d, J=7.2 Hz, 1H), 7.14 (t, J=7.7 Hz, 1H), 7.07 (d, J=8.2 Hz, 1H).

Synthesis of a Compound 9c

The compound 9b (6 g, 37.24 mmol), 2-methoxypyridine-4-boronic acid pinacolate (10.48 g, 44.68 mmol), bis(di-tert-butyl-4-dimethylaminophosphine)palladium chloride (Pd₁₃₂, 60 mg, 0.52 mmol), and potassium carbonate (15.42 g, 111.72 mmol) were added to toluene (100 ml) and water (20 ml) in a 250 ml one-mouth flask. A mixture obtained above was stirred for a reaction at 90° C. for 12 hours under the protection of nitrogen. After cooling to room temperature, water and ethyl acetate were added for extraction twice, and organic phases were combined. After a solvent was spun out, a residue was separated by column chromatography to obtain 2.7 g of a white solid with a yield of 31%. ¹H NMR (400 MHz, Chloroform-d) δ 8.23 (dd, J=7.6, 1.4 Hz, 1H), 8.19 (d, J=7.7 Hz, 1H), 7.76 (d, J=5.2 Hz, 1H), 7.43-7.35 (m, 2H), 7.32 (dd, J=9.3, 3.5 Hz, 2H), 7.28 (s, 1H), 7.20-7.16 (m, 3H), 7.08 (dd, J=6.5, 3.1 Hz, 2H), 6.57 (dd, J=5.2, 1.4 Hz, 1H), 6.42 (s, 1H), 3.84 (s, 3H).

Synthesis of a Compound 9d

The compound 9c (5 g, 14.28 mmol) was added to a mixed solvent of hydrobromic acid (10 ml) and acetic acid (30 ml) in a 100 ml one-mouth flask. A mixture obtained above was stirred for a reaction at 90° C. for 6 hours under the protection of nitrogen. 50 ml of water was added to the one-mouth flask until a light green solid was precipitated out, and the solid was filtered and dried to obtain 3.5 g of a product with a yield of 71%. Note: The product with poor solubility was directly used in the next step without identification.

Synthesis of a Compound 9e

The compound 9d (3.6 g, 10.7 mmol) was dissolved in a mixed solvent of phosphorus oxychloride (40 ml) and dichlorobenzene (4 ml) in a 100 ml one-mouth flask. A mixture obtained above was stirred for a reaction at 90° C. for 6 hours under the protection of nitrogen. A reaction solution was slowly poured into 200 ml of ice water, and potassium carbonate was added to adjust the pH to neutral. 100 ml of ethyl acetate was added for extraction twice, and organic phases were combined. After a solvent was spun out, a residue was separated by column chromatography to obtain 3.4 g of a white solid with a yield of 89%. ¹H NMR (400 MHz, Chloroform-d) δ 8.26 (d, J=6.5 Hz, 1H), 8.19 (d, J=7.9 Hz, 1H), 8.01 (d, J=4.9 Hz, 1H), 7.41 (dt, J=14.9, 7.3 Hz, 2H), 7.32 (dd, J=16.7, 8.9 Hz, 3H), 7.25-7.20 (m, 3H), 7.09 (d, J=7.6 Hz, 2H), 6.99 (s, 1H), 6.94 (dd, J=5.1, 1.4 Hz, 1H).

Synthesis of a Compound 9f

The 100 compound 9e (3.4 g, 9.58 mmol), a borate intermediate 9e-1 (4 g, 11.49 ml) (synthesized with reference to a patent CN110872325A), potassium carbonate (3.97 g, 28.74 mmol), and tetrakis(triphenylphosphine)palladium (140 mg) were dissolved in a mixed solvent of 1,4-dioxane (50 ml) and water (10 ml) in a 100 ml one-mouth flask. A mixture obtained above was stirred for a reaction at 100° C. for 12 hours under the protection of nitrogen. A reaction solution was extracted twice with 100 ml of ethyl acetate. An organic phase was spin-dried, and a residue was separated by column chromatography to obtain 5.9 g of a foamy white solid product with a yield of 79%.

¹H NMR (400 MHz, Chloroform-d) δ 8.56 (s, 1H), 8.39 (d, J=5.0 Hz, 1H), 8.26 (dd, J=5.6, 3.4 Hz, 1H), 8.19 (t, J=7.6 Hz, 2H), 8.03 (d, J=7.6 Hz, 3H), 7.97-7.91 (m, 2H), 7.58 (t, J=7.7 Hz, 1H), 7.47 (s, 1H), 7.40 (t, J=5.8 Hz, 4H), 7.34 (dd, J=14.8, 7.5 Hz, 2H), 7.23 (d, J=7.9 Hz, 1H), 7.12-6.99 (m, 9H), 3.88 (s, 3H), 1.40 (s, 18H).

Synthesis of a Compound 9g

The compound 9f (5.8 g, 9.58 mmol), pyridine hydrochloride (58 g, 0.5 mol), and o-dichlorobenzene (10 ml) were put into a 250 ml one-mouth flask. A mixture obtained above was stirred for a reaction at 100° C. for 10 hours under the protection of nitrogen. A reaction solution was extracted twice with 100 ml of ethyl acetate. An organic phase was spin-dried, and a residue was separated by column chromatography to obtain 5.4 g of a bright yellow powder with a yield of 94.9%. ¹H NMR (400 MHz, Chloroform-d) δ 8.56 (s, 1H), 8.39 (d, J=5.0 Hz, 1H), 8.26 (dd, J=5.6, 3.4 Hz, 1H), 8.19 (t, J=7.6 Hz, 2H), 8.03 (d, J=7.6 Hz, 3H), 7.98-7.90 (m, 2H), 7.58 (t, J=7.7 Hz, 1H), 7.55 (s, 4H), 7.47 (s, 1H), 7.40 (t, J=5.8 Hz, 4H), 7.34 (dd, J=14.8, 7.5 Hz, 2H), 7.23 (d, J=7.9 Hz, 1H), 7.10-7.02 (m, 6H), 1.40 (s, 18H).

Synthesis of a Complex 9

The compound 9g (4.5 g, 5.8 mmol), potassium tetrachloroplatinate (3.6 g, 9 mmol), and tetrabutylammonium bromide (280 mg, 0.9 mmol) were dissolved in acetic acid (500 ml) in a 1,000 ml one-mouth flask. A mixture obtained above was stirred for a reaction at 135° C. for 72 hours under the protection of nitrogen. Water was added to a reaction solution until a solid was precipitated out, and the solid was filtered to obtain a crude product. Recrystallization was conducted with a mixture of dichloromethane and n-hexane at a ratio of 1:1 to obtain 3.5 g of an orange yellow powder with a yield of 61.9%. ¹H NMR (400 MHz, Chloroform-d) δ 8.78 (d, J=5.8 Hz, 1H), 8.31 (dd, J=9.1, 4.4 Hz, 2H), 8.23 (d, J=7.7 Hz, 1H), 8.13 (d, J=8.5 Hz, 1H), 7.80 (s, 1H), 7.64 (d, J=7.3 Hz, 2H), 7.60 (d, J=1.6 Hz, 2H), 7.47-7.40 (m, 5H), 7.36 (t, J=7.4 Hz, 1H), 7.34-7.27 (m, 3H), 7.20 (t, J=7.5 Hz, 4H), 7.10 (t, J=7.7 Hz, 2H), 6.95 (d, J=7.5 Hz, 1H), 6.76 (d, J=8.2 Hz, 1H), 1.45 (s, 18H). ESI-MS (m/z): 947.3 (M+1).

### EXAMPLE 2

Synthesis of a Complex 11

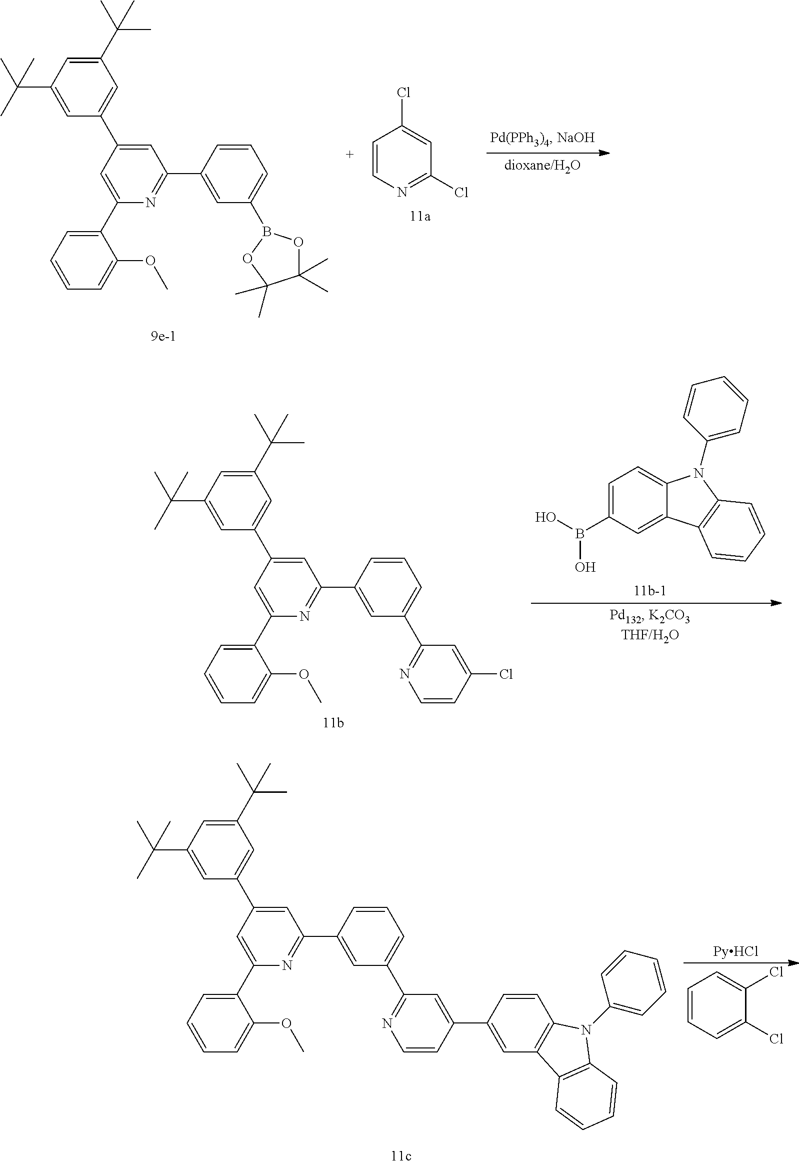

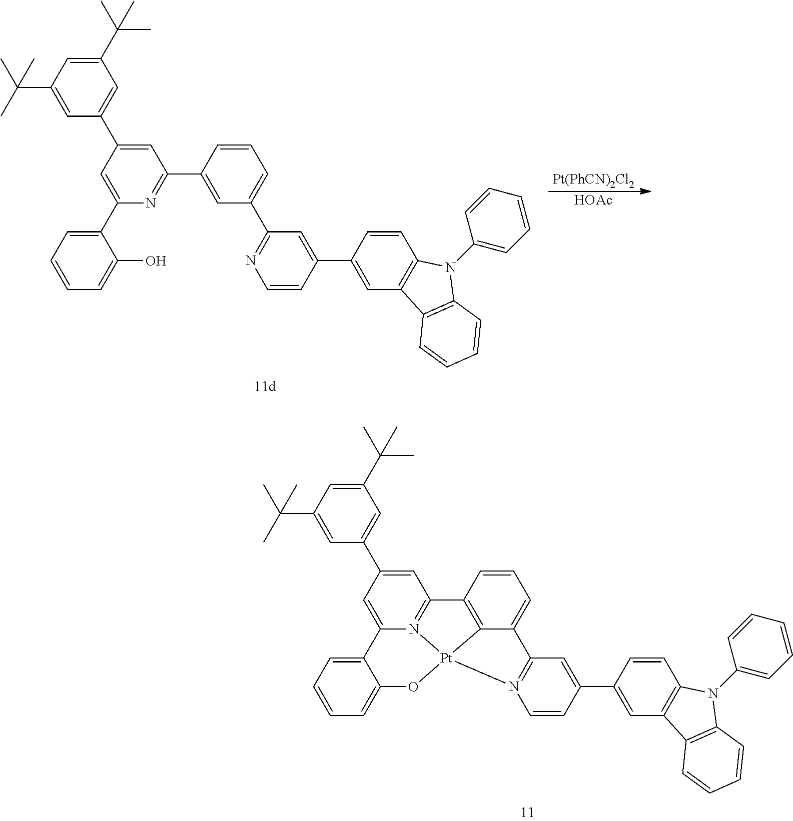

Synthesis of a Compound 11b

A compound 9e-1 (20.0 g, 33.92 mmol), a compound 11a (10.0 g, 67.84 mmol), tetrakis(triphenylphosphine)palladium (1.96 g, 1.69 mmol), sodium hydroxide (2.44 g, 61.1 mmol), dioxane (400 ml), and water (80 ml) were put into a 2 L three-mouth flask, and stirred for a reaction at 60° C. for 12 hours under the protection of nitrogen. After the reaction was completed, most of a solvent was spin-dried, and then water and dichloromethane were added for extraction for 2 times. The solvent was spun out, and a residue was separated by column chromatography to obtain 14 g of a yellow white solid with a yield of 73.68%. ¹H NMR (400 MHz, Chloroform-d) δ 8.69 (s, 1H), 8.61 (d, J=5.3 Hz, 1H), 8.23 (d, J=7.8 Hz, 1H), 8.12-8.01 (m, 3H), 7.91 (d, J=1.4 Hz, 1H), 7.85 (d, J=1.7 Hz, 1H), 7.62 (s, 1H), 7.55 (s, 3H), 7.47-7.39 (m, 1H), 7.30-7.26 (m, 1H), 7.16 (s, 1H), 7.06 (d, J=8.1 Hz, 1H), 3.92 (s, 3H), 1.41 (s, 18H).

Synthesis of a Compound 11c

The compound 11b (5.0 g, 8.91 mmol), a compound 11b-1 (3.83 g, 13.36 mmol), bis(di-tert-butyl-4-dimethylaminophosphine)palladium chloride (Pd₁₃₂, 0.19 g, 0.26 mmol), potassium carbonate (3.69 g, 26.7 mmol), tetrahydrofuran (125 ml), and water (25 ml) were put into a 250 ml one-mouth flask for a reaction at 70° C. for 12 hours under the protection of nitrogen. After the reaction was completed, most of a solvent was spin-dried, and then water and dichloromethane were added for extraction for 2 times. The solvent was spun out, and a residue was separated by column chromatography to obtain 6.0 g of a white solid with a yield of 87.72%. ¹H NMR (400 MHz, Chloroform-d) δ 8.82-8.75 (m, 2H), 8.51 (s, 1H), 8.28-8.20 (m, 2H), 8.18 (d, J=6.1 Hz, 2H), 8.10-8.02 (m, 2H), 7.96 (d, J=1.4 Hz, 1H), 7.78 (dd, J=8.6, 1.7 Hz, 1H), 7.70-7.58 (m, 6H), 7.58-7.47 (m, 5H), 7.47-7.37 (m, 3H), 7.34 (s, 1H), 7.14 (t, J=7.5 Hz, 1H), 7.05 (d, J=8.2 Hz, 1H), 3.91 (s, 3H), 1.41 (s, 18H).

Synthesis of a Compound 11d

The compound 11c (10.0 g, 13.03 mmol, 1.0 eq), pyridine hydrochloride (100 g), and o-dichlorobenzene (10 ml) were put into a 500 ml one-mouth flask for a reaction at 200° C. for 8 hours under the protection of nitrogen. After the reaction was completed, dichloromethane was used for extraction twice. A solvent was spun out, and a residue was separated by column chromatography to obtain 9.0 g of a yellow solid with a yield of 91.6%. ¹H NMR (400 MHz, Chloroform-d) δ 8.79 (d, J=5.1 Hz, 1H), 8.71 (s, 1H), 8.55 (s, 1H), 8.23 (dd, J=16.6, 7.6 Hz, 3H), 8.11 (d, J=7.8 Hz, 1H), 8.05 (s, 1H), 7.96 (d, J=8.6 Hz, 2H), 7.81 (dd, J=8.6, 1.7 Hz, 1H), 7.71 (t, J=7.8 Hz, 1H), 7.68-7.57 (m, 6H), 7.53 (dd, J=9.1, 5.1 Hz, 4H), 7.45 (d, J=6.0 Hz, 2H), 7.34 (d, J=7.3 Hz, 2H), 7.09 (d, J=8.1 Hz, 1H), 6.98 (s, 1H), 1.42 (s, 18H).

Synthesis of a Complex 11

The compound 11d (2.0 g, 2.65 mmol, 1.0 eq), bis(benzonitrile)dichloroplatinum (1.5 g, 3.18 mmol, 1.2 eq), and acetic acid (200 ml) were put into a 500 ml one-mouth flask for a reaction at 130° C. for 24 hours under the protection of nitrogen. After the reaction was completed, an excess amount of deionized water was added until a solid was precipitated out. Suction filtration was conducted, and the solid was dissolved in dichloromethane. A solvent was spun out, and a residue was separated by column chromatography to obtain 1.5 g of a yellow solid with a yield of 60.0%. ¹H NMR (400 MHz, Chloroform-d) δ 9.02 (d, J=6.1 Hz, 1H), 8.49 (s, 1H), 8.24 (d, J=8.1 Hz, 2H), 8.06 (d, J=8.2 Hz, 1H), 7.97 (s, 1H), 7.80-7.69 (m, 2H), 7.68-7.39 (m, 16H), 7.36 (t, J=7.2 Hz, 1H), 7.23 (d, J=7.5 Hz, 1H), 6.73 (s, 1H), 1.45 (s, 18H). ESI-MS (m/z): 948.3 (M+1).

### EXAMPLE 3

Synthesis of a Complex 16

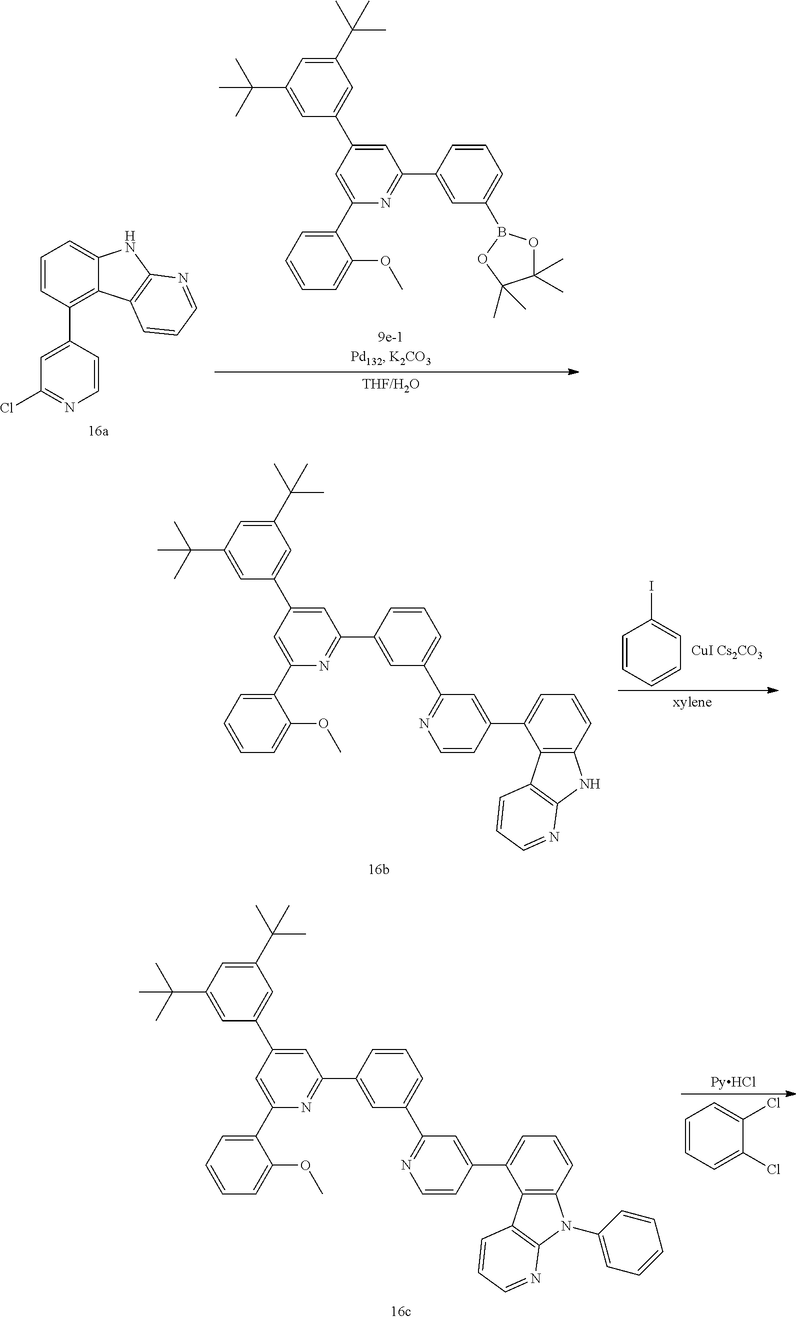

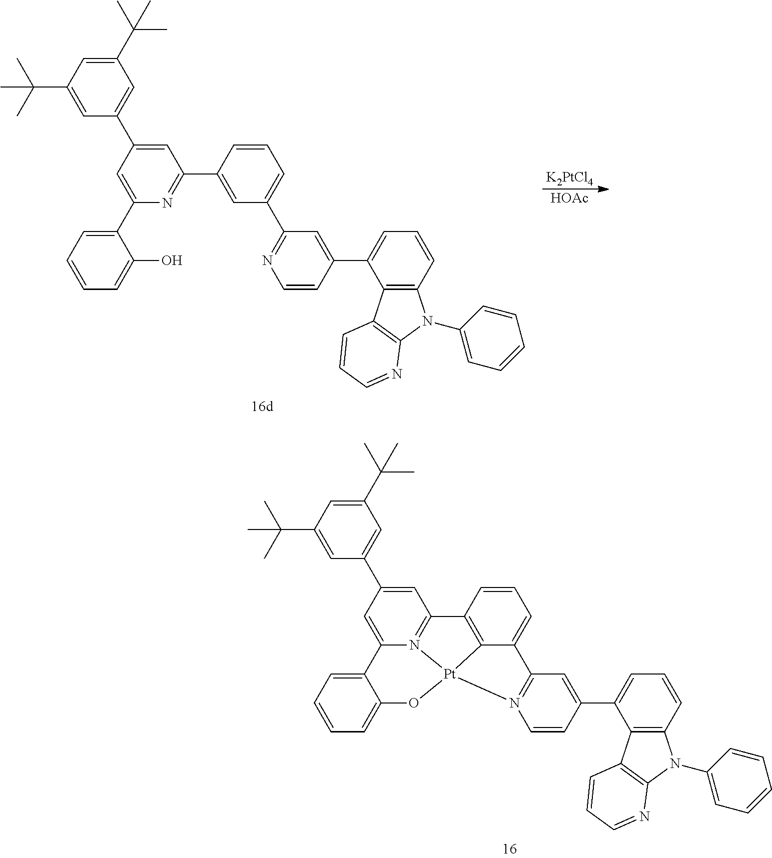

Synthesis of a Compound 16b

A compound 16a (2.02 g, 7.23 mmol) (customized and purchased), a compound 9e-1 (5.0 g, 8.68 mmol), bis(di-tert-butyl-4-dimethylaminophosphine)palladium chloride (Pd₁₃₂, 0.1 g, 0.14 mmol), potassium carbonate (3.0 g, 21.69 mmol), and a mixture of tetrahydrofuran (50 ml) and water (10 ml) were put into a 250 ml one-mouth flask for a reaction at 80° C. for 12 hours under the protection of nitrogen. After the reaction was completed, most of a solvent was spin-dried, and then water and dichloromethane were added for extraction for 2 times. The solvent was spun out, and a residue was separated by column chromatography to obtain 3.4 g of a white solid with a yield of 68%. ¹H NMR (400 MHz, Chloroform-d) δ 8.92 (d, J=5.0 Hz, 1H), 8.82 (s, 1H), 8.24 (d, J=7.4 Hz, 1H), 8.21-8.15 (m, 2H), 8.02 (dd, J=8.9, 1.5 Hz, 2H), 7.93 (d, J=1.4 Hz, 1H), 7.71 (d, J=7.9 Hz, 1H), 7.68-7.62 (m, 3H), 7.60-7.49 (m, 7H), 7.49-7.42 (m, 2H), 7.42-7.34 (m, 2H), 7.30 (t, J=7.1 Hz, 1H), 7.22 (dd, J=6.9, 1.3 Hz, 1H), 7.08 (dt, J=16.4, 7.9 Hz, 3H), 3.89 (s, 3H), 1.41 (s, 18H).

Synthesis of a Compound 16c

The compound 16b (2.5 g, 3.61 mmol), cuprous iodide (345 mg, 1.81 mmol), cesium carbonate (3.53 g, 10.83 mmol), copper (115 mg, 1.81 mmol), phenanthroline (651 mg, 3.61 mmol), and xylene (50 ml) were put into a 250 ml three-mouth flask, and stirred for a reaction at 160° C. for 30 hours under the protection of nitrogen. After the reaction was completed, a mixture obtained was treated, subjected to suction filtration directly, and rinsed with ethyl acetate. A solvent was spun out, and a residue was separated by column chromatography to obtain 2.2 g of a white solid with a yield of 79.42%. ¹H NMR (400 MHz, Chloroform-d) δ 8.78 (d, J=4.4 Hz, 1H), 8.61 (dd, J=3.4, 1.9 Hz, 1H), 8.41 (dd, J=6.9, 1.9 Hz, 1H), 8.27 (d, J=2.2 Hz, 1H), 8.21 (t, J=1.9 Hz, 1H), 8.10 (d, J=2.2 Hz, 1H), 7.95-7.87 (m, 4H), 7.84 (dd, J=7.2, 1.2 Hz, 1H), 7.70 (dd, J=6.4, 1.2 Hz, 1H), 7.64 (dd, J=8.9, 8.2 Hz, 1H), 7.54-7.45 (m, 3H), 7.45-7.37 (m, 5H), 7.37 (ddt, J=6.3, 4.9, 1.3 Hz, 3H), 7.27 (dd, J=6.9, 3.4 Hz, 1H), 7.15 (dd, J=8.7, 7.5, 1.2 Hz, 1H), 6.90 (dd, J=7.7, 1.2 Hz, 1H), 3.90 (s, 3H), 1.35 (s, 18H).

Synthesis of a Compound 16d

The compound 16c (3.8 g, 4.9 mmol), pyridine hydrochloride (38 g), and o-dichlorobenzene (3.8 ml) were put into a 250 ml one-mouth flask for a reaction at 200° C. for 8 hours under the protection of nitrogen. After the reaction was completed, dichloromethane was used for extraction twice. After a solvent was spun out, recrystallization was conducted on an obtained crude with a mixture of ethyl acetate and methanol at a ratio of 6:1. 2.9 g of a light yellow solid with a yield of 78.40% was obtained. ¹H NMR (400 MHz, Chloroform-d) δ 8.78 (d, J=4.4 Hz, 1H), 8.61 (dd, J=3.4, 1.9 Hz, 1H), 8.41 (dd, J=6.9, 1.9 Hz, 1H), 8.27 (d, J=2.2 Hz, 1H), 8.21 (t, J=1.9 Hz, 1H), 8.11 (d, J=2.2 Hz, 1H), 7.99 (dd, J=8.7, 1.2 Hz, 1H), 7.93-7.81 (m, 4H), 7.70 (dd, J=6.4, 1.2 Hz, 1H), 7.64 (dd, J=8.9, 8.2 Hz, 1H), 7.54-7.45 (m, 3H), 7.45-7.35 (m, 6H), 7.39-7.32 (m, 2H), 7.31-7.24 (m, 1H), 7.28-7.20 (m, 1H), 7.03-6.94 (m, 2H), 1.35 (s, 18H).

Synthesis of a Complex 16

The compound 16d (2.5 g, 3.4 mmol), potassium tetrachloroplatinate (1.7 g, 4.1 mmol), tetrabutylammonium bromide (109 mg, 0.34 mmol), and acetic acid (250 ml) were put into a 100 ml one-mouth flask for a reaction at 130° C. for 48 hours under the protection of nitrogen. After the reaction was completed, an excess amount of deionized water was added until a solid was precipitated out. Suction filtration was conducted, and the solid was dissolved in dichloromethane. A solvent was spun out, and a residue was separated by column chromatography to obtain 2.40 g of a red solid with a yield of 75%. ¹H NMR (400 MHz, Chloroform-d) δ 9.45-9.39 (m, 1H), 9.35-9.28 (m, 1H), 8.97-8.92 (m, 1H), 8.61 (dd, J=3.4, 1.9 Hz, 1H), 8.47-8.36 (m, 3H), 8.36-8.30 (m, 1H), 8.30-8.24 (m, 1H), 8.12 (d, J=2.2 Hz, 1H), 7.89 (t, J=7.8 Hz, 1H), 7.84 (dd, J=7.2, 1.1 Hz, 1H), 7.70 (dd, J=6.4, 1.2 Hz, 1H), 7.54-7.24 (m, 11H), 7.19-7.10 (m, 1H), 7.00 (dd, J=7.5, 1.3 Hz, 1H), 1.35 (s, 18H). ESI-MS (m/z): 948.3 (M+1)

### EXAMPLE 4

Synthesis of a Complex 30

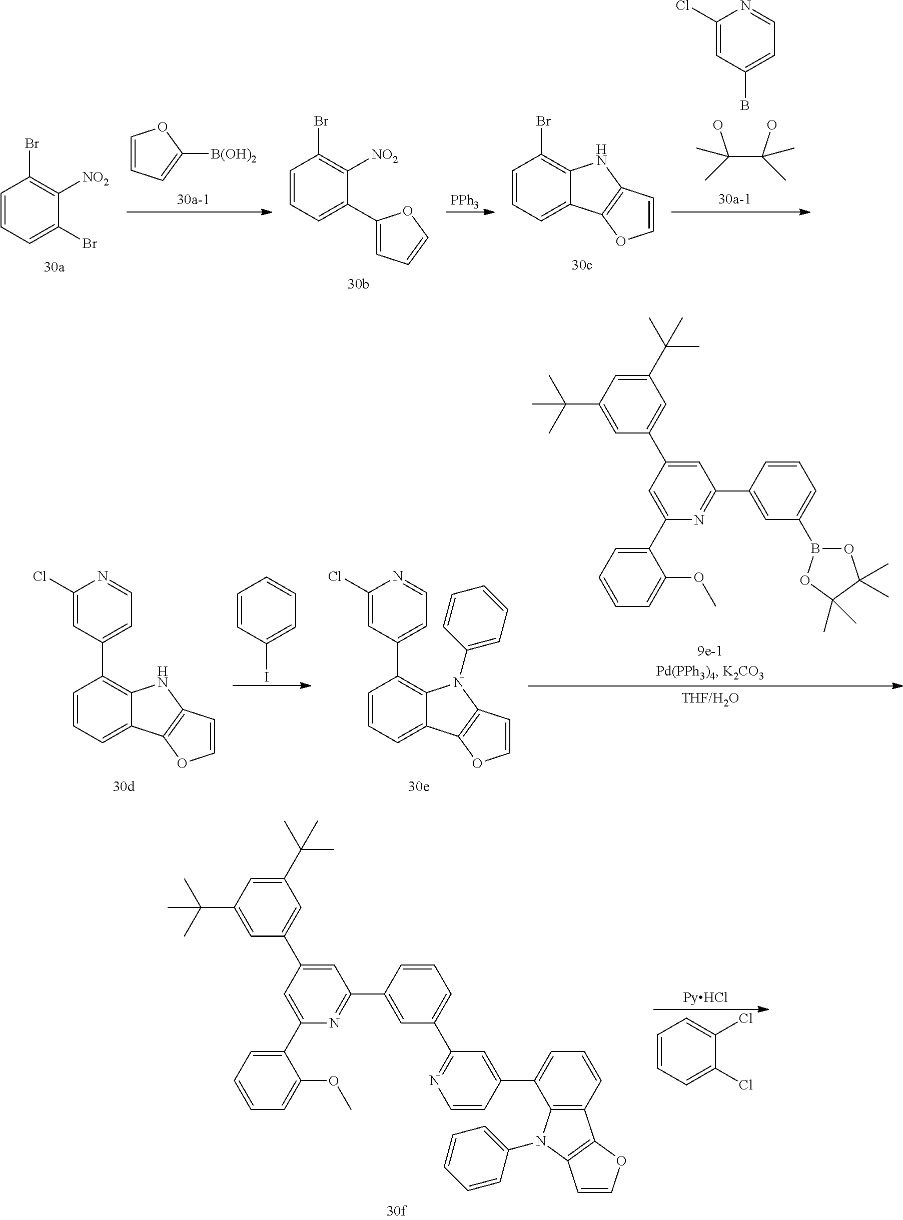

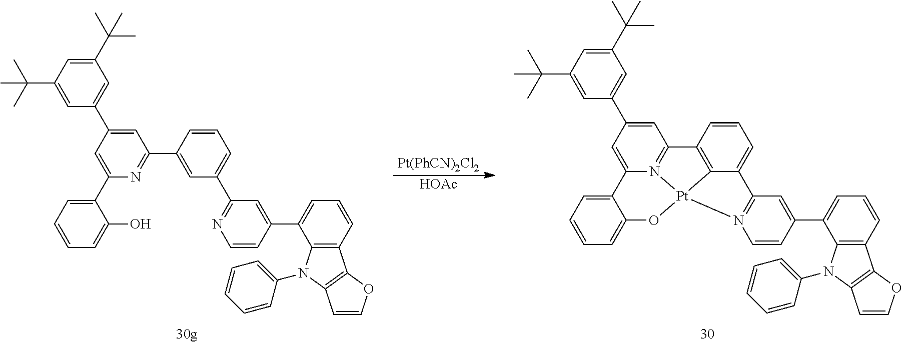

Synthesis of a Compound 30b

An intermediate 30a (20.0 g, 71.4 mmol), a compound 30a-1 (8.0 g, 71.4 ml), potassium carbonate (29.56 g, 214.2 mmol), and tetrakis(triphenylphosphine)palladium (400 mg) were dissolved in a mixed solvent of 1,4-dioxane (400 ml) and water (100 ml) in a 1 L flask. A mixture obtained above was stirred at 95° C. for 12 hours. A reaction solution was extracted twice with 200 ml of ethyl acetate. An organic phase was spin-dried, and a residue was separated by column chromatography to obtain 12.1 g of a foamy white solid with a yield of 63.3%. ¹H NMR (400 MHz, Chloroform-d) δ 7.83-7.74 (m, 2H), 7.66 (t, J=1.5 Hz, 1H), 7.54 (dd, J=8.8, 7.7 Hz, 1H), 7.19 (dd, J=4.8, 1.7 Hz, 1H), 6.72 (dd, J=4.8, 1.3 Hz, 1H).

Synthesis of a Compound 30c

The intermediate 30b (10.0 g, 37.3 mmol) and triphenylphosphine (29.36 g, 111.9 mmol) were added to 1,2-dichlorobenzene (120 ml) in a 250 ml flask, and stirred at 165° C. for 12 hours. After a reaction was completed, a reaction solution was extracted with water and dichloromethane. A solvent was spun out, and a residue was separated by column chromatography to obtain 5.99 g of a yellow solid with a yield of 68.1%. ¹H NMR (400 MHz, Chloroform-d) δ 9.92 (s, 1H), 8.08 (dd, J=9.0, 1.1 Hz, 1H), 7.80 (s, 1H), 7.57 (dd, J=7.8, 1.2 Hz, 1H), 7.24 (dd, J=9.0, 7.9 Hz, 1H), 6.85 (d, J=0.7 Hz, 1H).

Synthesis of a Compound 30d

The compound 30c (7.0 g, 29.65 mmol), a compound 30b-1 (8.5 g, 35.58 mmol), bis(di-tert-butyl-4-dimethylaminophosphine)palladium chloride (0.42 g, 0.593 mmol), potassium carbonate (10.23 g, 74.12 mmol), dioxane (100 ml), and water (20 ml) were put into a 250 ml one-mouth flask for a reaction at 110° C. for 12 hours under the protection of nitrogen. After the reaction was completed, most of a solvent was spin-dried, and then water and dichloromethane (50 ml) were added for extraction for 3 times. The solvent was spun out, and a residue was separated by column chromatography to obtain 5.13 g of a white solid with a yield of 64.45%. ¹H NMR (400 MHz, Chloroform-d) δ 8.43 (d, J=4.5 Hz, 1H), 8.14-8.09 (m, 1H), 7.87-7.79 (m, 2H), 7.65 (d, J=2.2 Hz, 1H), 7.51 (dd, J=4.5, 2.3 Hz, 1H), 7.37 (dd, J=9.0, 7.5 Hz, 1H), 6.83 (s, 1H).

Synthesis of a Compound 30e

The compound 30d (5 g, 18.6 mmol), iodobenzene (11.3 g, 55.8 mmol), cuprous iodide (0.35 g, 1.86 mmol), a copper powder (0.12 g, 1.86 mmol), 1,2-cyclohexenediamine (0.64 g, 5.58 mmol), and xylene (150 ml) were added to a 250 ml one-mouth flask, and stirred for a reaction at 100° C. for 12 hours under the protection of nitrogen. After the reaction was completed, toluene (100 ml) was used for rinsing twice. A solvent was spun out, and a residue was separated by column chromatography to obtain 4.31 g of a colorless oily product with a yield of 67.1%. ¹H NMR (400 MHz, Chloroform-d) δ 8.43 (d, J=4.5 Hz, 1H), 8.19 (dd, J=8.4, 0.7 Hz, 1H), 7.87 (d, J=1.3 Hz, 1H), 7.67 (d, J=2.3 Hz, 1H), 7.66-7.62 (m, 1H), 7.55-7.46 (m, 3H), 7.43-7.33 (m, 4H), 7.02 (d, J=1.3 Hz, 1H).

Synthesis of a Compound 30f

The compound 30e (4.0 g, 11.6 mmol), a borate intermediate 9e-1 (8.01 g, 13.92 ml) (synthesized with reference to a patent CN110872325A), potassium carbonate (4.80 g, 34.8 mmol), and tetrakis(triphenylphosphine)palladium (80 mg) were dissolved in a mixed solvent of 1,4-dioxane (80 ml) and water (20 ml) in a 250 ml one-mouth flask. A mixture obtained above was stirred for a reaction at 100° C. for 12 hours under the protection of nitrogen. A reaction solution was extracted twice with 100 ml of ethyl acetate. An organic phase was spin-dried, and a residue was separated by column chromatography to obtain 6.7 g of a white solid with a yield of 76.2%. ¹H NMR (400 MHz, Chloroform-d) δ 8.75 (d, J=4.6 Hz, 1H), 8.26 (d, J=2.3 Hz, 1H), 8.23-8.16 (m, 2H), 8.10 (d, J=2.2 Hz, 1H), 7.95-7.85 (m, 5H), 7.67 (dd, J=7.0, 0.7 Hz, 1H), 7.67-7.60 (m, 1H), 7.53-7.46 (m, 3H), 7.42 (d, J=2.1 Hz, 2H), 7.42-7.34 (m, 6H), 7.15 (ddd, J=8.6, 7.5, 1.1 Hz, 1H), 7.02 (d, J=1.3 Hz, 1H), 6.90 (dd, J=7.7, 1.2 Hz, 1H), 3.90 (s, 3H), 1.35 (s, 18H).

Synthesis of a Compound 30g

The compound 30f (6.5 g, 8.57 mmol), pyridine hydrochloride (65 g), and o-dichlorobenzene (6.5 ml) were put into a 250 ml one-mouth flask for a reaction at 200° C. for 8 hours under the protection of nitrogen. After the reaction was completed, dichloromethane was used for extraction twice. A solvent was spun out, and a residue was separated by column chromatography to obtain 6.2 g of a yellow solid with a yield of 97.3%. ¹H NMR (400 MHz, Chloroform-d) δ 8.75 (d, J=4.6 Hz, 1H), 8.26 (d, J=2.3 Hz, 1H), 8.23-8.16 (m, 2H), 8.11 (d, J=2.2 Hz, 1H), 7.99 (dd, J=8.8, 1.3 Hz, 1H), 7.92-7.83 (m, 4H), 7.67 (dd, J=7.0, 0.7 Hz, 1H), 7.67-7.60 (m, 1H), 7.53-7.46 (m, 3H), 7.42 (d, J=2.1 Hz, 2H), 7.42-7.34 (m, 6H), 7.25 (td, J=8.0, 1.3 Hz, 1H), 7.04-6.94 (m, 3H), 1.35 (s, 18H).

Synthesis of a Complex 30

The compound 30g (5.0 g, 6.72 mmol), bis(benzonitrile)dichloroplatinum (3.03 g, 8.06 mmol), and acetic acid (400 ml) were put into a 500 ml one-mouth flask for a reaction at 130° C. for 24 hours under the protection of nitrogen. After the reaction was completed, an excess amount of deionized water was added until a solid was precipitated out. Suction filtration was conducted, and the solid was dissolved in dichloromethane. A solvent was spun out, and a residue was separated by column chromatography to obtain 3.2 g of a yellow solid with a yield of 52.4%. ¹H NMR (400 MHz, Chloroform-d) δ 9.07 (d, J=8.9 Hz, 1H), 8.23 (d, J=2.1 Hz, 1H), 8.19 (dd, J=8.4, 0.7 Hz, 1H), 8.11 (d, J=2.1 Hz, 1H), 7.94 (dd, J=8.2, 1.2 Hz, 1H), 7.87 (d, J=1.4 Hz, 1H), 7.74 (d, J=2.2 Hz, 1H), 7.70 (dd, J=8.9, 2.3 Hz, 1H), 7.63-7.57 (m, 2H), 7.57-7.50 (m, 1H), 7.53-7.47 (m, 2H), 7.47 (t, J=7.5 Hz, 1H), 7.43-7.35 (m, 6H), 7.33-7.27 (m, 1H), 7.17 (dd, J=7.5, 1.2 Hz, 1H), 7.09 (ddd, J=8.4, 7.5, 1.3 Hz, 1H), 7.07 (s, 1H), 7.02 (d, J=1.3 Hz, 1H), 1.35 (s, 18H). ESI-MS (m/z): 938.3 (M+1)

### EXAMPLE 5

Synthesis of a Complex 47

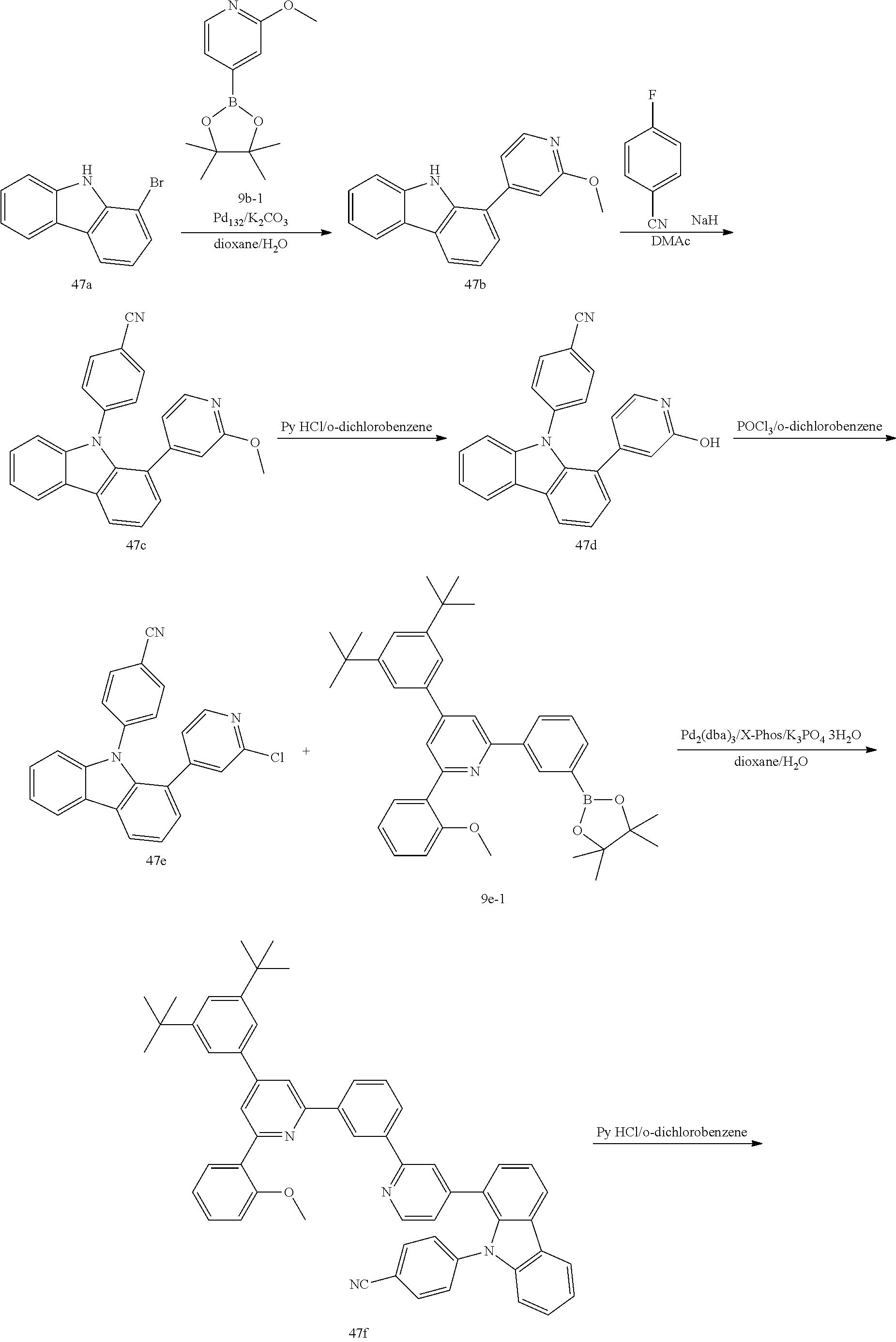

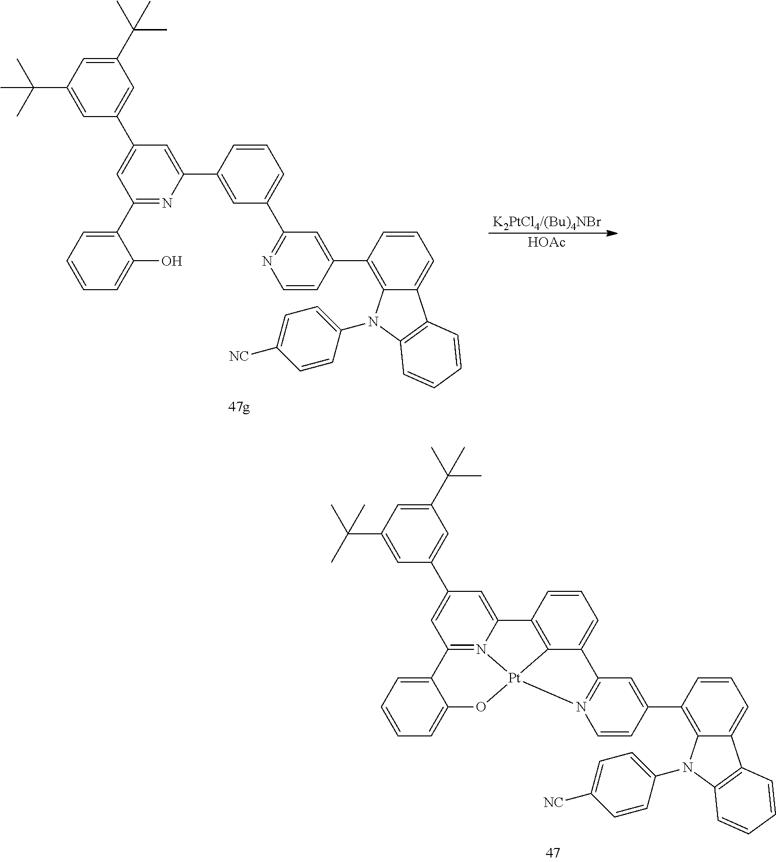

Synthesis of a Compound 47b

A mixture of a compound 47a (3.3 g, 13.41 mmol), a compound 9b-1 (4.1 g, 17.43 mmol), bis(di-tert-butyl-4-dimethylaminophosphine)palladium chloride (Pd₁₃₂, 0.094 g, 0.134 mmol), potassium carbonate (4.63 g, 33.52 mmol), dioxane (35 ml), and water (7 ml) was heated to 110° C., and stirred for a reaction for 12 hours under the protection of nitrogen. After the reaction was completed, most of a solvent was spun out, and then water and dichloromethane (50 ml) were added for extraction for 3 times. The solvent was spun out, and a residue was separated by column chromatography to obtain 6.4 g of a white solid with a yield of 86.9%. ¹H NMR (400 MHz, Chloroform-d) δ 8.52 (s, 1H), 8.31 (d, J=5.2 Hz, 1H), 8.15-8.09 (m, 2H), 7.46 (dd, J=9.1, 2.6 Hz, 3H), 7.33 (t, J=7.6 Hz, 1H), 7.30-7.25 (m, 1H), 7.20 (dd, J=5.3, 1.4 Hz, 1H), 7.08 (d, J=0.6 Hz, 1H), 4.03 (s, 3H).

Synthesis of a Compound 47c

A mixture of the compound 47b (3.0 g, 10.94 mmol), 4-fluorobenzonitrile (3.31 g, 27.34 mmol), sodium hydride (0.52 g, 21.87 mmol), and N,N-dimethylacetamide (30 ml) was heated to 110° C., and stirred for a reaction for 48 hours under the protection of nitrogen. After the reaction was completed, a mixture obtained was treated. A reaction solution was slowly dropped into ice water, and extracted with dichloromethanee (50 ml) for 3 times. Then, an organic phase was washed with water (50 ml) for 6 times. A solvent was spun out, and a residue was separated by column chromatography to obtain 9.2 g of a white solid with a yield of 74.6%. ¹H NMR (400 MHz, Chloroform-d) δ 8.20 (dd, J=15.1, 7.6 Hz, 2H), 7.86 (d, J=5.2 Hz, 1H), 7.48 (d, J=8.2 Hz, 2H), 7.45-7.43 (m, 1H), 7.42-7.40 (m, 1H), 7.38-7.34 (m, 2H), 7.26 (d, J=8.1 Hz, 1H), 7.19 (d, J=8.2 Hz, 2H), 6.65-6.60 (m, 1H), 6.34 (s, 1H), 3.88 (s, 3H).

Synthesis of a Compound 47d

The compound 47c (9.0 g, 23.97 mmol), pyridine hydrochloride (90 g), and o-dichlorobenzene (9 ml) were put into a 500 ml one-mouth flask for a reaction at 200° C. for 8 hours under the protection of nitrogen. After the reaction was completed, an excess amount of water was added for suction filtration. A solid was washed with water for 2 times, beaten with methanol (50 ml) for 2 hours, and then beaten with ethyl acetate (50 ml) by heating reflux for 12 hours. 5.0 g of a white solid with a yield of 57.7% was obtained. ¹H NMR (400 MHz, Chloroform-d) δ 8.26 (dd, J=7.7, 1.2 Hz, 1H), 8.19 (d, J=7.6 Hz, 1H), 8.12 (d, J=4.9 Hz, 1H), 7.55 (d, J=8.5 Hz, 2H), 7.50-7.34 (m, 5H), 7.23 (d, J=8.2 Hz, 2H), 7.00 (dd, J=7.5, 2.5 Hz, 2H).

Synthesis of a Compound 47e

The compound 47d (5.0 g, 13.83 mmol), phosphorus oxychloride (50 ml), and o-dichlorobenzene (5 ml) were put into a 250 ml one-mouth flask for a reaction at 90° C. for 12 hours under the protection of nitrogen. After the reaction was completed, a reaction solution was slowly dropped into ice water, and extracted with dichloromethanee (50 ml) for 3 times. A solvent was spun out, and a residue was separated by column chromatography to obtain 4.1 g of a white solid with a yield of 78.1%. ¹H NMR (400 MHz, Chloroform-d) δ 8.26 (dd, J=7.7, 1.2 Hz, 1H), 8.19 (d, J=7.6 Hz, 1H), 8.12 (d, J=4.9 Hz, 1H), 7.56 (d, J=8.5 Hz, 2H), 7.45 (t, J=7.6 Hz, 3H), 7.37 (ddd, J=8.6, 7.6, 1.1 Hz, 2H), 7.23 (d, J=8.2 Hz, 2H), 7.00 (dd, J=7.5, 2.5 Hz, 2H).

Synthesis of a Compound 47f

The compound 47e (1.8 g, 4.74 mmol, 1.0 eq), a compound 9e-1 (5.45 g, 9.48 mmol, 2.0 eq), tris(dibenzylideneacetone)dipalladium (0.22 g, 0.24 mmol, 0.05 eq), 2-dicyclohexylphosphino-2′,4′,6′-triisopropylbiphenyl (0.11 g, 0.24 mmol), potassium phosphate trihydrate (3.78 g, 14.22 mmol), dioxane (20 ml), and water (4 ml) were subjected to a reaction at 90° C. for 12 hours under the protection of nitrogen. After the reaction was completed, a mixture obtained was treated. Most of a solvent was spin-dried, and then water and dichloromethane (30 ml) were added for extraction for 3 times. The solvent was spun out, and a residue was separated by column chromatography to obtain 4.2 g of a white solid with a yield of 56%. ¹H NMR (400 MHz, Chloroform-d) δ 8.55-8.47 (m, 2H), 8.25 (dd, J=5.1, 3.9 Hz, 1H), 8.17 (dd, J=15.5, 7.5 Hz, 2H), 8.06 (d, J=7.7 Hz, 1H), 7.99-7.91 (m, 3H), 7.62 (t, J=7.8 Hz, 1H), 7.54 (s, 3H), 7.48-7.44 (m, 2H), 7.44-7.36 (m, 4H), 7.33 (d, J=8.8 Hz, 2H), 7.20 (s, 1H), 7.15 (s, 1H), 7.13 (d, J=1.6 Hz, 1H), 7.01 (dd, J=15.6, 8.0 Hz, 2H), 3.86 (s, 3H), 1.40 (s, 18H).

Synthesis of a Compound 47g

The compound 47f (4.1 g, 5.17 mmol), pyridine hydrochloride (40 g), and o-dichlorobenzene (4 ml) were put into a 250 ml one-mouth flask for a reaction at 200° C. for 8 hours under the protection of nitrogen. After the reaction was completed, water and dichloromethane (40 ml) were added for extraction for 3 times. A solvent was spun out, and a residue was separated by column chromatography to obtain 3.8 g of a yellow solid. ¹H NMR (400 MHz, Chloroform-d) δ 8.51 (d, J=5.0 Hz, 1H), 8.38 (s, 1H), 8.27 (t, J=4.5 Hz, 1H), 8.22 (d, J=7.4 Hz, 1H), 8.05 (dd, J=13.5, 6.6 Hz, 3H), 7.92 (d, J=7.6 Hz, 2H), 7.67 (t, J=7.8 Hz, 1H), 7.59 (s, 1H), 7.53 (d, J=1.6 Hz, 2H), 7.48 (d, J=4.5 Hz, 2H), 7.46-7.35 (m, 3H), 7.33-7.22 (m, 7H), 7.15 (d, J=4.8 Hz, 1H), 6.94 (t, J=7.5 Hz, 1H), 6.77 (d, J=8.2 Hz, 1H), 1.42 (s, 18H).

Synthesis of a Complex 47

The compound 47g (0.5 g, 0.64 mmol), potassium tetrachloroplatinate (0.32 g, 0.77 mmol), tetrabutylammonium bromide (0.01 g, 0.032 mmol), and acetic acid (50 ml) were put into a 100 ml one-mouth flask for a reaction at 130° C. for 48 hours under the protection of nitrogen. An excess amount of water was added, and suction filtration was conducted. A solid was washed with water for 2 times, and then dissolved in dichloromethane. A solvent was spun out, and a residue was separated by column chromatography to obtain 0.2 g of a yellow solid with a yield of 22.9%. ¹H NMR (400 MHz, Chloroform-d) δ 8.93 (d, J=5.7 Hz, 1H), 8.34 (s, 1H), 8.29 (d, J=4.6 Hz, 1H), 8.22 (d, J=7.8 Hz, 1H), 8.13 (d, J=8.1 Hz, 1H), 7.82 (s, 1H), 7.66 (s, 1H), 7.61 (d, J=5.1 Hz, 2H), 7.52-7.37 (m, 10H), 7.29 (s, 2H), 7.23 (d, J=6.2 Hz, 4H), 6.77 (s, 1H), 1.44 (d, J=8.0 Hz, 18H). ESI-MS (m/z): 972.3 (M+1)

### EXAMPLE 6

Synthesis of a Complex 50

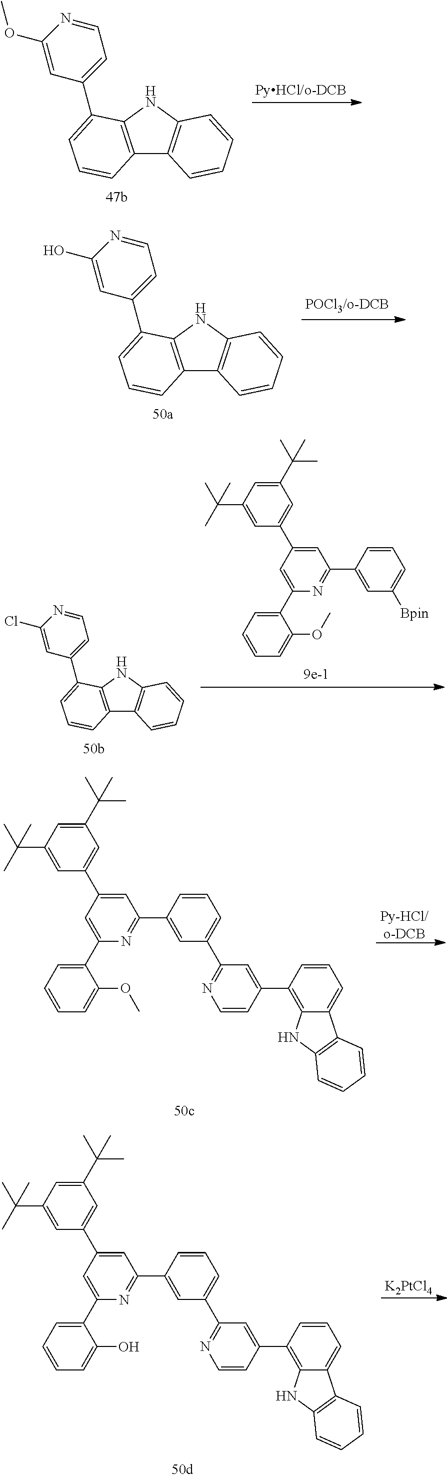

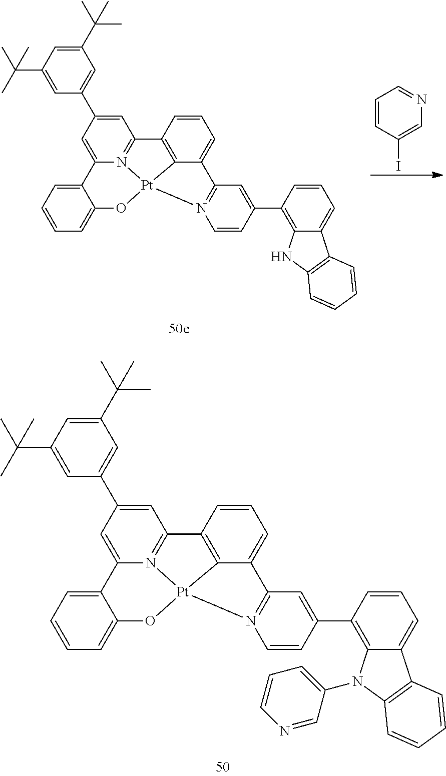

Synthesis of a Compound 50a

The compound 47b (4.5 g, 1.6 mmol), pyridine hydrochloride (45 g), and o-dichlorobenzene (4.5 ml) were put into a 250 ml one-mouth flask for a reaction at 180° C. for 3.5 hours under the protection of nitrogen. After the reaction was completed, cooling was conducted to room temperature. Water and dichloromethane were added and stirred for 30 minutes. After liquid separation, an organic layer was collected to obtain a crude product. The crude product was beaten with n-hexane to obtain 4.3 g of a yellow solid. ¹H NMR (400 MHz, DMSO-d6) δ 11.22 (s, 1H), 8.15 (dd, J=18.5, 7.7 Hz, 2H), 7.59-7.52 (m, 2H), 7.46-7.36 (m, 2H), 7.24 (t, J=7.6 Hz, 1H), 7.20-7.13 (m, 1H), 6.67 (d, J=1.2 Hz, 1H), 6.52 (dd, J=6.7, 1.7 Hz, 1H).

Synthesis of a Compound 50b

The compound 50a (4.5 g, 1.7 mmol), phosphorus oxychloride (50 ml), and o-dichlorobenzene (3 ml) were added to a 250 ml one-mouth flask for a reaction at 100° C. for 18 hours under the protection of nitrogen. After the reaction was completed, cooling was conducted to room temperature. Next, ice water was added and stirred to completely quench the phosphorus oxychloride. Then, a reaction solution was extracted with dichloromethane to obtain a crude product. The crude product was beaten with n-hexane to obtain 4.2 g of a yellow solid. ¹H NMR (400 MHz, Chloroform-d) δ 8.59 (s, 1H), 8.49 (dd, J=5.1, 0.6 Hz, 1H), 8.14 (dd, J=17.2, 7.5 Hz, 2H), 7.70-7.65 (m, 1H), 7.55 (dd, J=5.1, 1.5 Hz, 1H), 7.53-7.42 (m, 3H), 7.35 (t, J=7.6 Hz, 1H), 7.32-7.27 (m, 1H).

Synthesis of a Compound 50c

The compound 50b (6.0 g, 21.5 mmol), a compound 9e-1 (13.5 g, 23.5 mmol), tris(dibenzylideneacetone)dipalladium (2.15 g, 10% eq), 2-dicyclohexylphosphino-2′,4′,6′-triisopropylbiphenyl (2.15 g, 4.3 mmol), potassium phosphate trihydrate (17 g, 64.5 mmol), toluene (100 ml), ethanol (60 ml), and H₂O (40 ml) were put into a 250 ml one-mouth flask for a reaction at 110° C. for 12 hours under the protection of nitrogen. After the reaction was completed, cooling was conducted to room temperature. Suction filtration was conducted to obtain a filtrate. An organic phase was removed by rotary evaporation. A reaction solution was extracted, and dichloromethane layers were combined. A solvent was spun out, and a residue was separated by column chromatography to obtain 8 g of a brown white solid with a yield of 53.6%. ¹H NMR (400 MHz, Chloroform-d) δ 8.85 (d, J=4.6 Hz, 1H), 8.80 (s, 1H), 8.68 (s, 1H), 8.22 (d, J=7.9 Hz, 1H), 8.14 (dd, J=13.8, 8.3 Hz, 4H), 8.05-7.99 (m, 2H), 7.92 (d, J=1.4 Hz, 1H), 7.67-7.51 (m, 7H), 7.43-7.36 (m, 4H), 7.26 (s, 1H), 7.06 (dd, J=15.3, 7.9 Hz, 2H), 3.88 (s, 3H), 1.42 (s, 18H).

Synthesis of a Compound 50d

The compound 50c (10.0 g, 14.2 mmol), pyridine hydrochloride (100 g), and o-dichlorobenzene (10 ml) were put into a 250 ml one-mouth flask, and heated to 180° C. for a reaction for 5 hours under the replacement of nitrogen. After the reaction was completed, cooling was conducted to room temperature. Water and dichloromethane were added and stirred for 30 minutes. After liquid separation, an organic layer was collected. A solvent was spun out, and a residue was separated by column chromatography to obtain 9.0 g of a yellow solid with a yield of 87.0%. ¹H NMR (400 MHz, Chloroform-d) δ 8.83 (d, J=5.0 Hz, 1H), 8.75 (d, J=7.0 Hz, 1H), 8.67 (s, 1H), 8.19-8.09 (m, 4H), 8.07-8.00 (m, 2H), 7.93 (d, J=7.9 Hz, 1H), 7.88 (s, 1H), 7.61 (dt, J=7.6, 6.8 Hz, 3H), 7.53 (t, J=5.2 Hz, 3H), 7.47 (d, J=8.1 Hz, 1H), 7.41-7.29 (m, 4H), 7.27-7.23 (m, 1H), 7.00-6.91 (m, 2H), 1.42 (s, 18H).

Synthesis of a Compound 50e

The compound 50d (1.01 g, 1.47 mmol.), potassium tetrachloroplatinate (840 mg, 1.98 mmol), tetrabutylammonium bromide (120 mg), and acetic acid (30 ml) were put into a 250 ml one-mouth flask, and heated to 180° C. for a reaction for 2 days under the replacement of nitrogen. After the reaction was completed, cooling was conducted to room temperature. A small amount of water was added to precipitate a compound. Suction filtration was conducted to obtain a solid. The solid was washed with methanol for three times. A crude product was separated by a silica gel column to obtain 800 mg of a yellow solid with a yield of 97.5%. ¹H NMR (400 MHz, tetrahydrofuran-d4) δ 10.71 (s, 1H), 9.11 (d, J=5.6 Hz, 1H), 8.48 (s, 1H), 8.36 (s, 1H), 8.27-8.19 (m, 2H), 8.14 (d, J=7.7 Hz, 1H), 8.01 (s, 1H), 7.79 (dd, J=10.6, 4.7 Hz, 5H), 7.71-7.60 (m, 2H), 7.49 (d, J=8.1 Hz, 1H), 7.43-7.32 (m, 2H), 7.29 (d, J=5.4 Hz, 2H), 7.22 (dd, J=13.8, 7.2 Hz, 2H), 6.64 (d, J=5.9 Hz, 1H), 1.46 (s, 18H).

Synthesis of a Complex 50

The compound 50e (4.35 g, 5.0 mmol), 4-iodopyridine (3.10 g, 15.0 mmol), a copper powder (300 mg, 15.0 mmol.), cuprous iodide (1.0 g, 15.0 mmol), Cs₂CO₃ (5.0 g, 15.0 mmol.), phenanthroline (1.0 g), and xylene (100 ml) were put into a 250 ml one-mouth flask for a reflux reaction for 48 hours under the protection of nitrogen. After the reaction was completed, cooling was conducted to room temperature. A small amount of water was added to precipitate a compound 50. Suction filtration was conducted to obtain a solid. The solid was washed with methanol for several times. A crude product was obtained, and a residue was separated by column chromatography to obtain 2.3 g of an orange yellow solid with a yield of 48.9%. ¹H NMR (400 MHz, Chloroform-d) δ 8.81 (d, J=5.6 Hz, 1H), 8.57 (s, 1H), 8.32-8.28 (m, 2H), 8.22 (d, J=7.7 Hz, 2H), 8.10 (d, J=8.2 Hz, 1H), 7.77 (s, 1H), 7.61 (dd, J=13.0, 1.6 Hz, 4H), 7.44 (dq, J=17.2, 7.3 Hz, 8H), 7.29 (d, J=7.5 Hz, 2H), 7.24-7.19 (m, 1H), 7.13 (d, J=3.8 Hz, 1H), 7.01 (s, 1H), 6.76 (d, J=5.5 Hz, 1H), 1.45 (s, 18H). ESI-MS (m/z): 948.3 (M+1)

It should be understood by those skilled in the art that the above preparation methods are merely several exemplary examples, and improvements may be conducted by those skilled in the art to obtain other compound structures of the present invention.

### EXAMPLE 7

About 5.0 mg of fully dried samples of the platinum complexes 9, 11, 16, 30, 47, and 50 were separately weighed under the atmosphere of nitrogen. The samples were measured at a heating scanning rate of 10° C./min in a scanning range of 25-800° C. to have a thermal decomposition temperature of 437° C., 541° C., 448° C., 441° C., 452° C., and 451° C. (the temperature corresponding to 5% of thermal weight loss), respectively, indicating that such complexes have excellent thermal stability.

### EXAMPLE 8

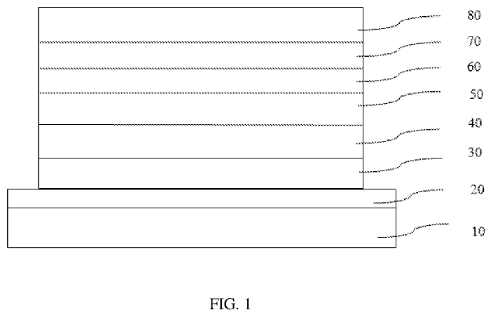

An organic light-emitting diode was prepared by using the complex luminescent material of the present invention. The structure of the device is as shown in FIG. 1.

First, a transparent conductive ITO glass substrate 10 (with an anode 20) was washed with a detergent solution, deionized water, ethanol, acetone, and deionized water in sequence, and then treated with oxygen plasma for 30 seconds.

Next, HATCN with a thickness of 10 nm was evaporated on the ITO to serve as a hole injection layer 30.

Next, a compound HT was evaporated to form a hole transport layer 40 with a thickness of 40 nm.

Next, a light-emitting layer 50 with a thickness of 20 nm was evaporated on the hole transport layer. The light-emitting layer was obtained by mixing and doping the platinum complex 9 (20%) and CBP (80%).

Then, AlQ₃ with a thickness of 40 nm was evaporated on the light-emitting layer to serve as an electron transport layer 60.

At last, LiF with a thickness of 1 nm and Al with a thickness of 100 nm were evaporated to serve as an electron injection layer 70 and a device cathode 80, respectively.

### EXAMPLE 9

An organic light-emitting diode was prepared by using the complex 11 instead of the complex 9 according to the method described in Example 7.

### EXAMPLE 10

An organic light-emitting diode was prepared by using the complex 16 instead of the complex 9 according to the method described in Example 7.

### EXAMPLE 11

An organic light-emitting diode was prepared by using the complex 30 instead of the complex 9 according to the method described in Example 7.

### EXAMPLE 12

An organic light-emitting diode was prepared by using the complex 47 instead of the complex 9 according to the method described in Example 7.

### EXAMPLE 13

An organic light-emitting diode was prepared by using the complex 50 instead of the complex 9 according to the method described in Example 7.

### COMPARATIVE EXAMPLE 1

An organic light-emitting diode was prepared by using a complex Ref-1(CN110872325A) instead of the complex 9 according to the method described in Example 7.

### COMPARATIVE EXAMPLE 2

An organic light-emitting diode was prepared by using a complex Ref-2(Chem. Sci., 2014, 5, 4819) instead of the complex 9 according to the method described in Example 7.

### COMPARATIVE EXAMPLE 3

An organic light-emitting diode was prepared by using a complex Ref-3(CN110872325A) instead of the complex 9 according to the method described in Example 3.

### COMPARATIVE EXAMPLE 4

An organic light-emitting diode was prepared by using a complex Ref-4(CN110872325A) instead of the complex 9 according to the method described in Example 3.

The HATCN, the HT, the AlQ₃, the Ref-1, the Ref-2, the Ref-3, the Ref-4, and the CBP in the devices have the following structural formulas:

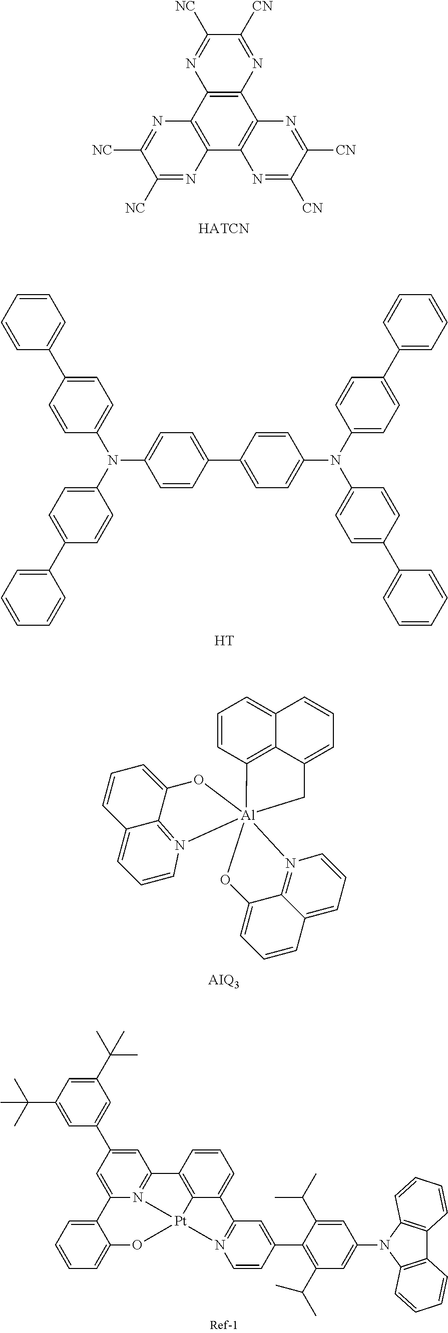

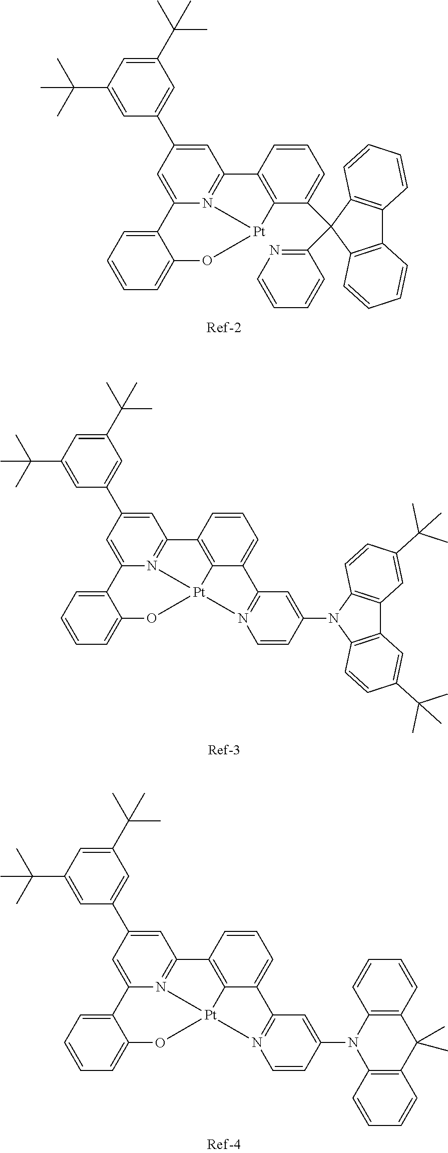

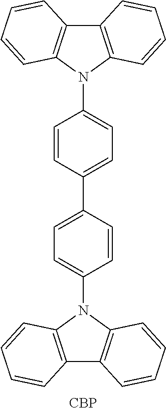

Device properties of the organic electroluminescent devices in Example 3, Comparative Example 1, Comparative Example 2, Comparative Example 3, and Comparative Example 4 at a current density of 20 mA/cm² are listed in Table 1:

TABLE 1

| | | | | Device |
|---|---|---|---|---|
| Device | | Driving | Luminescence | service |
| number | Complex | voltage | efficiency | life (LT95) |
| Example 8 | Complex 9 | 1 | 1 | 1 |
| Example 9 | Complex 11 | 0.9 | 0.98 | 0.71 |
| Example 10 | Complex 16 | 1 | 0.98 | 0.52 |
| Example 11 | Complex 30 | 0.97 | 0.99 | 0.62 |
| Example 12 | Complex 47 | 0.96 | 0.97 | 0.54 |
| Example 13 | Complex 50 | 0.93 | 0.98 | 0.56 |
| Comparative | Ref-1 | 1.1 | 0.95 | 0.35 |
| Example 1 | | | | |
| Comparative | Ref-2 | 1.1 | 0.91 | 0.20 |
| Example 2 | | | | |
| Comparative | Ref-3 | 1.07 | 0.72 | 0.36 |
| Example 3 | | | | |
| Comparative | Ref-4 | 1.05 | 0.83 | 0.07 |
| Example 4 | | | | |

Note:
The device properties are tested with Example 8 as a reference, and each indicator is set as 1. LT95 refers to the time when the brightness of a device is reduced to 95% of an initial brightness (10,000 cd/m²).

From the data in Table 1, it can be seen that when the platinum complex materials of the present invention are applied in the organic light-emitting diodes under the same conditions, lower driving voltage and higher luminescence efficiency are achieved. In addition, the device service life of the organic light-emitting diodes based on the complexes of the present invention is significantly longer than that of the complex materials in the comparative examples. Requirements of the display industry for luminescent materials can be met, and a great industrial prospect is achieved.

The various embodiments above are merely used as examples, and are not intended to limit the scope of the present invention. Other materials and structures may be used to replace the various materials and structures in the present invention without departing from the spirit of the present invention. It should be understood that various modifications and changes may be made by those skilled in the art according to the concept of the present invention without creative effort. Therefore, all technical solutions obtained by technical persons after analysis, inference, or partial research on the basis of existing technologies shall fall within the protection scope defined by the claims.

1. An ONCN quadridentate ligand-containing platinum complex, having the structure as shown in a formula (I): 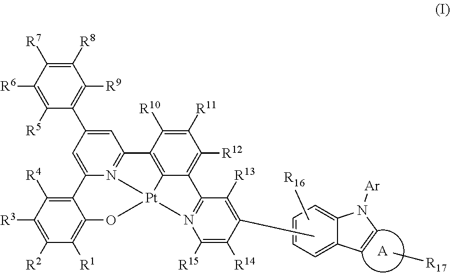
wherein each of R¹ to R¹⁷ is independently selected from hydrogen, deuterium, halogen, amino, carbonyl, carboxyl, thioalkyl, cyano, sulfonyl, phosphino, substituted or unsubstituted alkyl containing 1-20 carbon atoms, substituted or unsubstituted cycloalkyl containing 3-20 carbon atoms, substituted or unsubstituted alkenyl containing 2-20 carbon atoms, substituted or unsubstituted alkoxyl containing 1-20 carbon atoms, substituted or unsubstituted aryl containing 6-30 carbon atoms, and substituted or unsubstituted heteroaryl containing 3-30 carbon atoms; or any two adjacent substituents are connected or condensed into a ring;
Ar is selected from substituted or unsubstituted aryl containing 6-30 carbon atoms and substituted or unsubstituted heteroaryl containing 3-30 carbon atoms;
A is a five-membered or six-membered aromatic ring or heteraromatic ring;
a heteroatom in the heteroaryl or heteroaromatic ring comprises one or more of N, S, and O;
and the “substituted” refers to substitution with halogen, amino, cyano, or C₁-C₄ alkyls.

2. The platinum complex according to claim 1, wherein each of the R¹ to R¹⁷ is independently selected from hydrogen, deuterium, halogen, amino, thioalkyl, cyano, substituted or unsubstituted alkyl containing 1-6 carbon atoms, substituted or unsubstituted cycloalkyl containing 3-6 carbon atoms, substituted or unsubstituted alkenyl containing 2-6 carbon atoms, substituted or unsubstituted alkoxyl containing 1-6 carbon atoms, substituted or unsubstituted aryl containing 6-12 carbon atoms, and substituted or unsubstituted heteroaryl containing 3-6 carbon atoms;
and the Ar is selected from substituted or unsubstituted aryl containing 6-12 carbon atoms and substituted or unsubstituted heteroaryl containing 3-12 carbon atoms.

3. The platinum complex according to claim 2, wherein each of the R¹ to R¹⁷ is independently selected from hydrogen, deuterium, halogen, C₁-C₄ alkyls, cyano, substituted or unsubstituted cycloalkyl containing 3-6 carbon atoms, substituted or unsubstituted aryl containing 6-12 carbon atoms, and substituted or unsubstituted heteroaryl containing 3-6 carbon atoms;
and the Ar is selected from substituted or unsubstituted aryl containing 6-12 carbon atoms and substituted or unsubstituted heteroaryl containing 3-12 carbon atoms.

4. The platinum metal complex according to claim 3, wherein each of the R¹ to R¹⁷ is independently selected from hydrogen, deuterium, methyl, isopropyl, isobutyl, tert-butyl, cyano, substituted or unsubstituted cyclopentyl, substituted or unsubstituted cyclohexyl, substituted or unsubstituted phenyl, substituted or unsubstituted pyridyl, substituted or unsubstituted pyrazinyl, and substituted or unsubstituted pyrimidinyl;
the Ar is selected from substituted or unsubstituted phenyl, substituted or unsubstituted pyridyl, substituted or unsubstituted pyrazinyl, and substituted or unsubstituted pyrimidinyl;
the A is selected from a benzene ring, a pyridine ring, a pyrazine ring, a pyrimidine ring, a thiophene ring, a furan ring, a pyrazole ring, and an imidazole ring;
and the “substituted” refers to substitution with halogen, cyano, or C₁-C₄ alkyls.

5. The platinum metal complex according to claim 4, wherein each of the R¹ to R¹⁷ is independently selected from hydrogen, deuterium, methyl, tert-butyl, cyano, substituted or unsubstituted cyclopentyl, substituted or unsubstituted cyclohexyl, or substituted or unsubstituted phenyl;
the Ar is selected from substituted or unsubstituted phenyl and substituted or unsubstituted pyridyl;
and the A is selected from a benzene ring, a pyridine ring, a pyrazine ring, and a pyrimidine ring.

6. The platinum metal complex according to claim 5, wherein each of the R¹ to R¹⁷ is independently selected from hydrogen, deuterium, and tert-butyl;
the Ar is selected from phenyl, cyanophenyl, and pyridyl;
and the A is selected from a benzene ring, a pyridine ring, a pyrazine ring, and a pyrimidine ring.

7. The platinum metal complex according to claim 6, wherein among the R¹ to R¹⁷, R⁶ and R⁸ are tert-butyl, and the other groups are hydrogen;
the Ar is selected from phenyl and cyanophenyl;
and the A is selected from a benzene ring and a pyridine ring.

8. The platinum metal complex according to claim 1, being one of the following compounds:

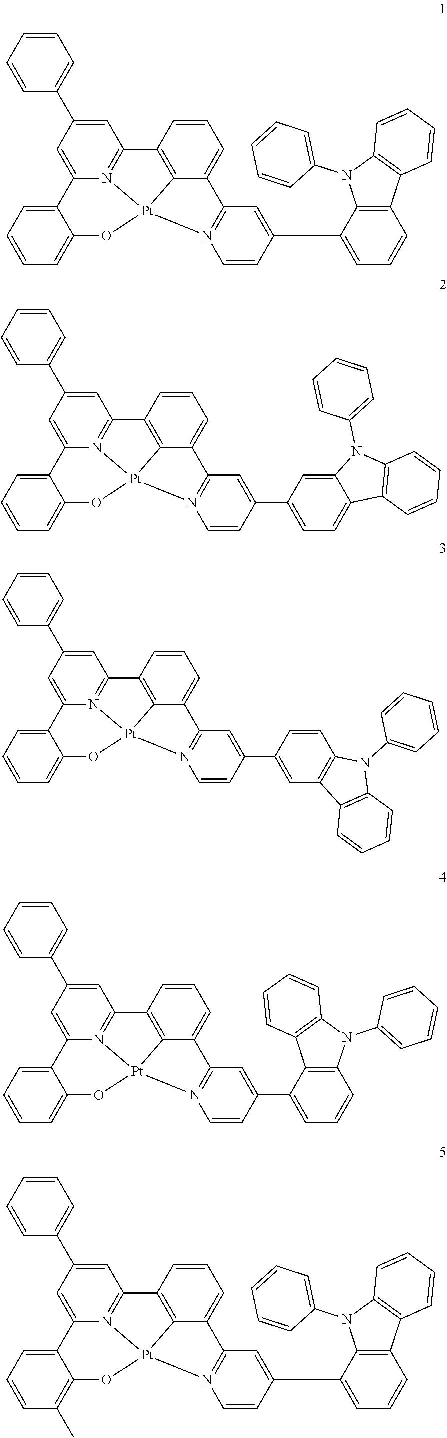

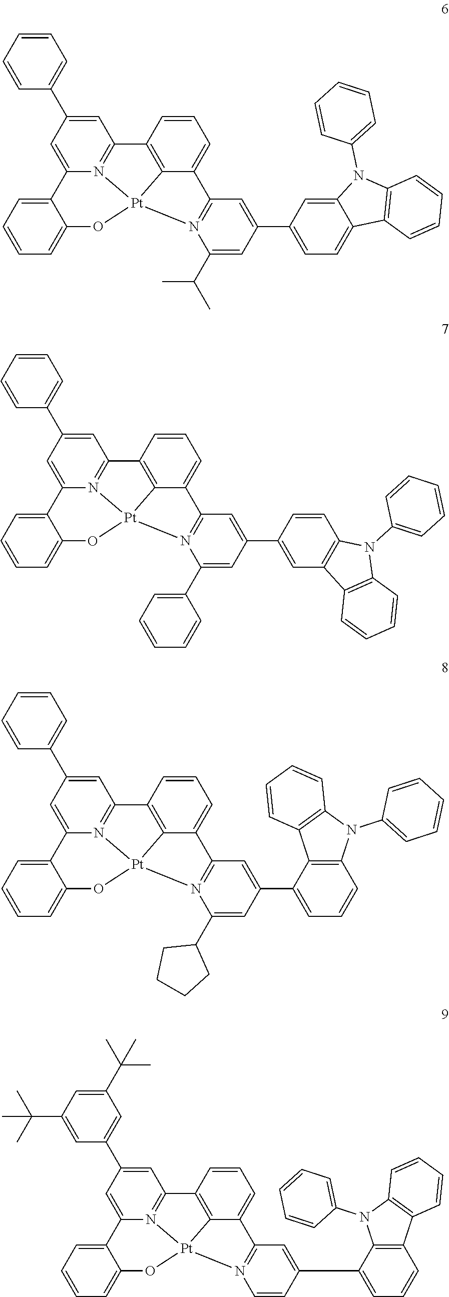

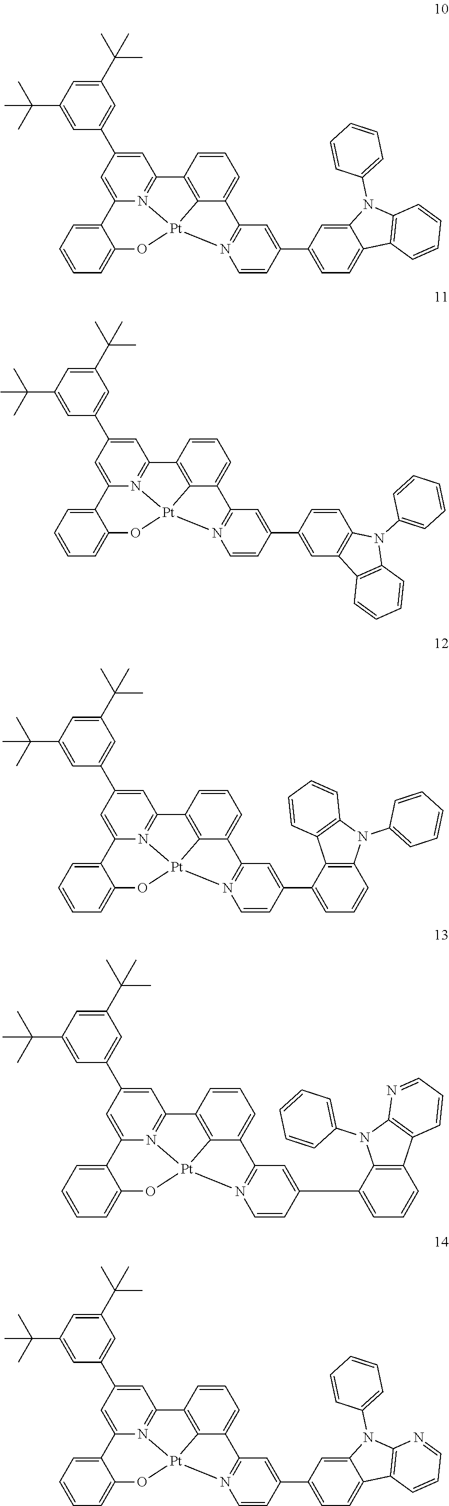

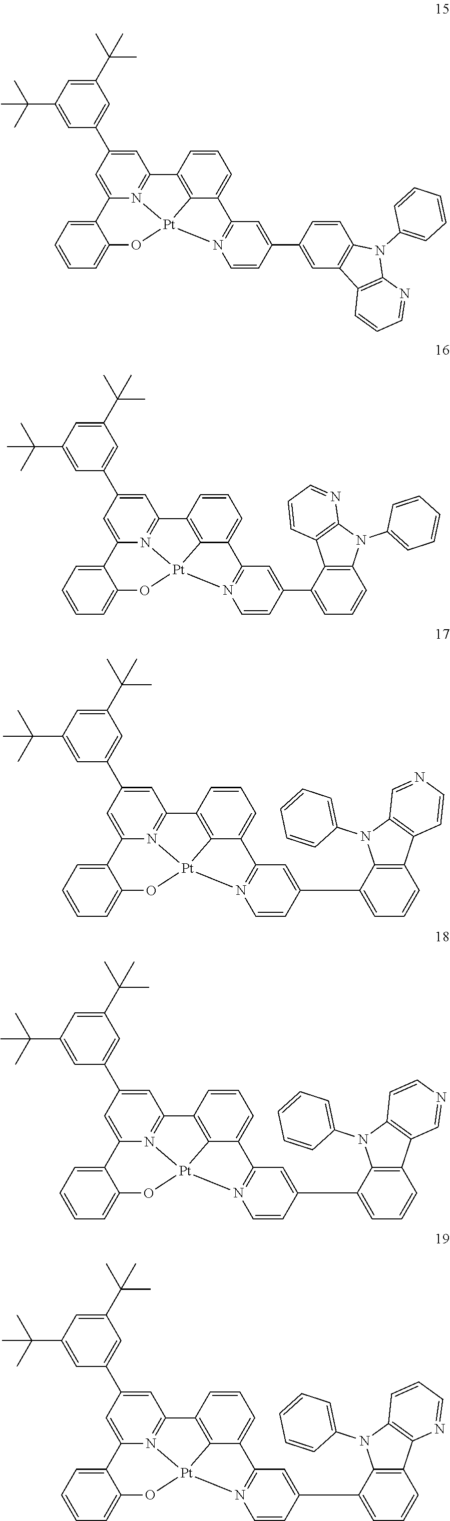

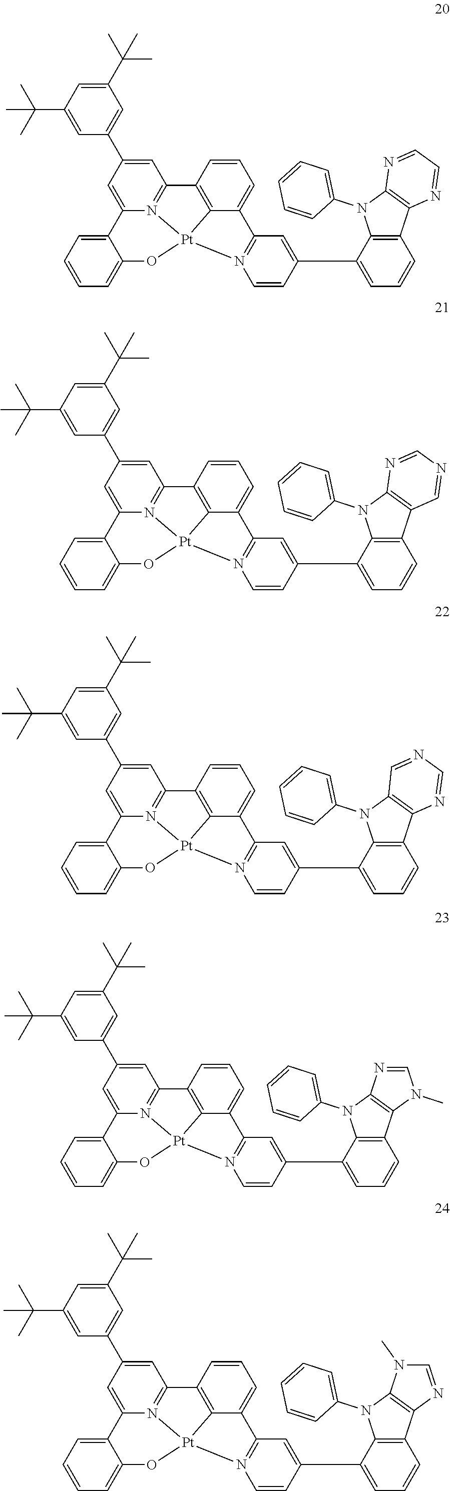

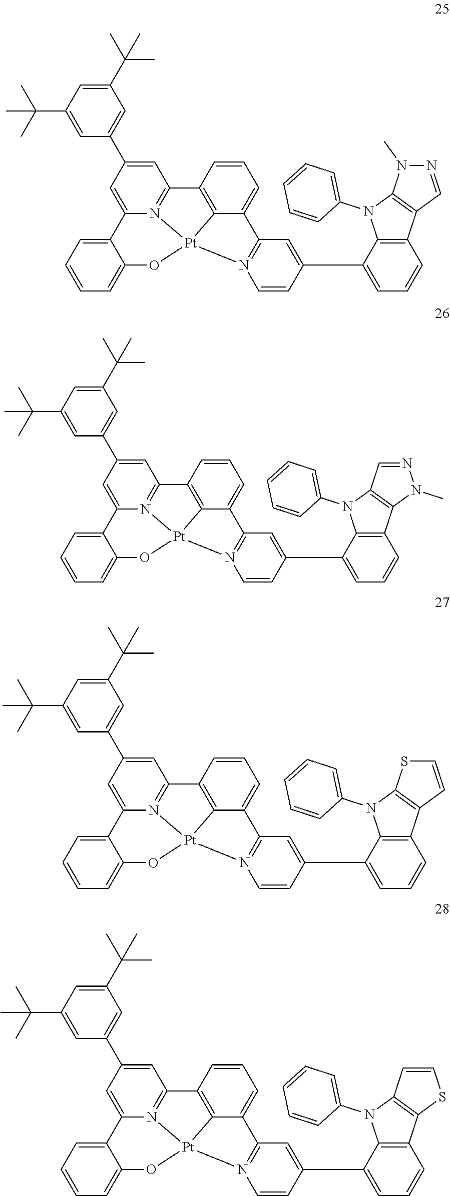

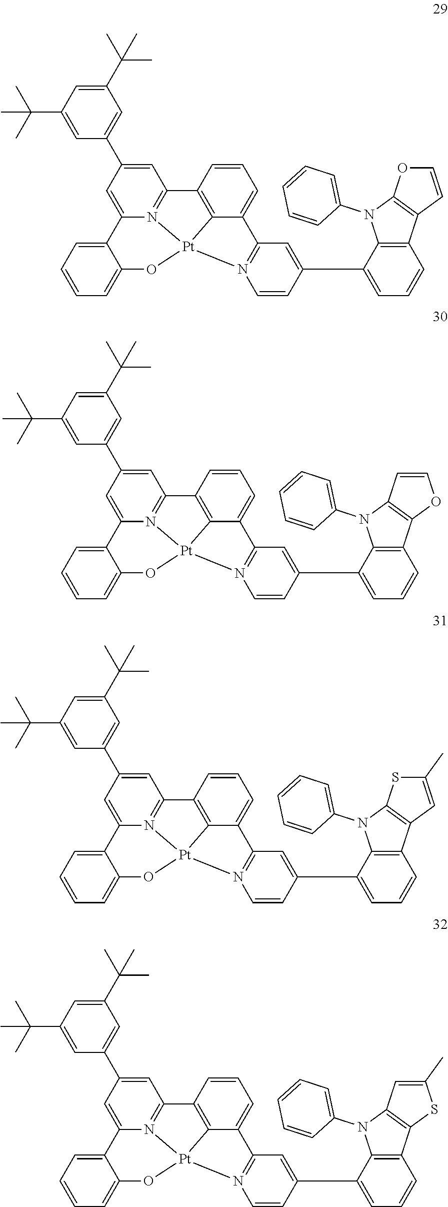

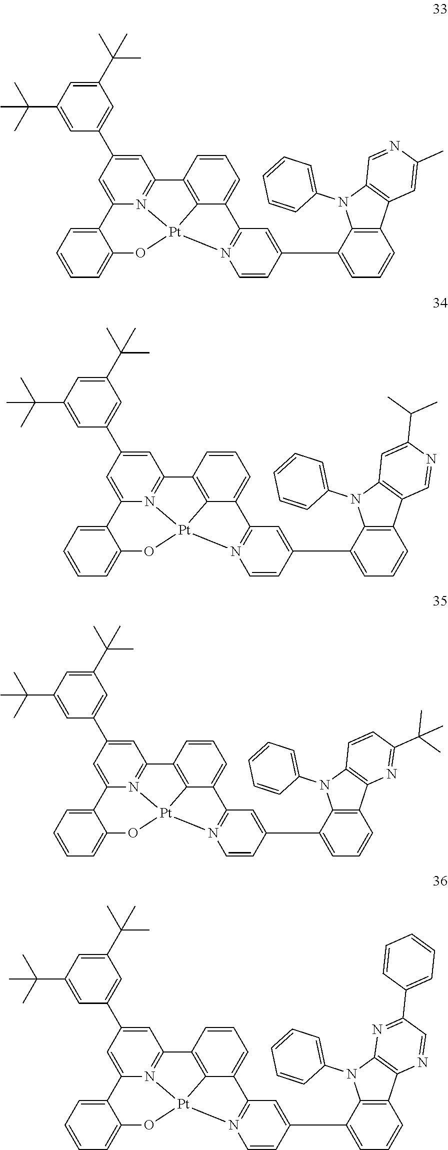

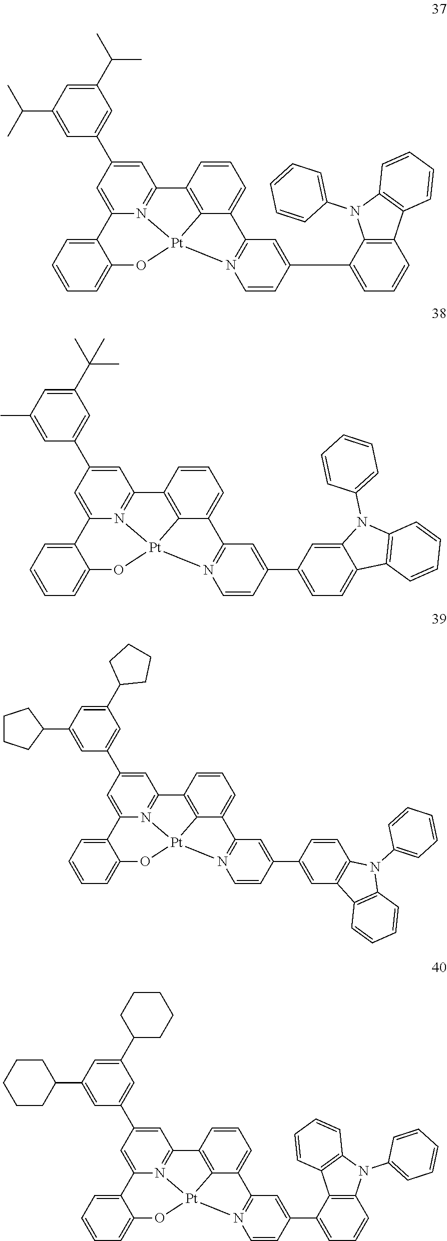

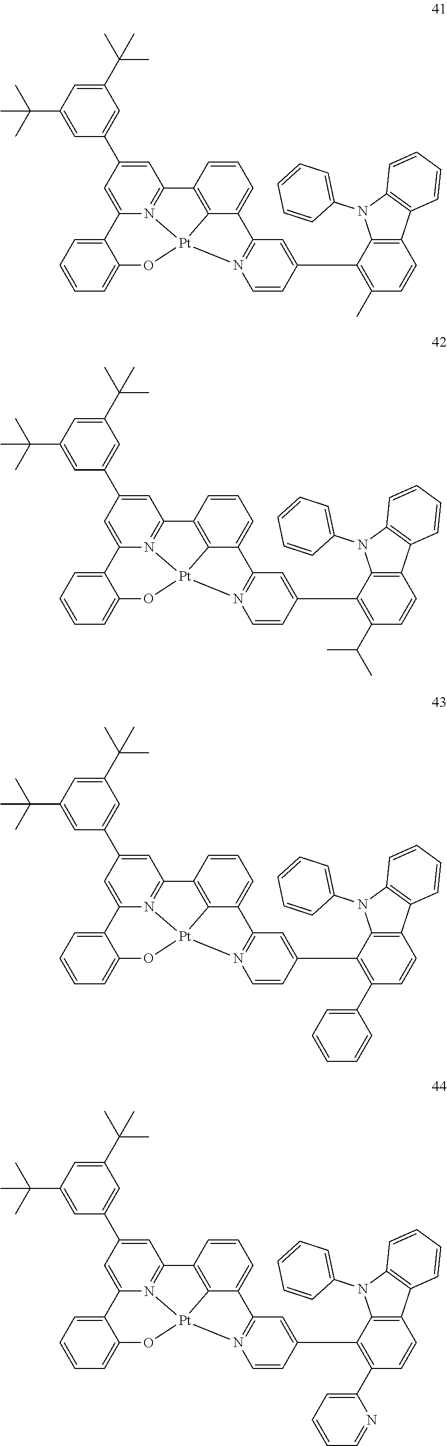

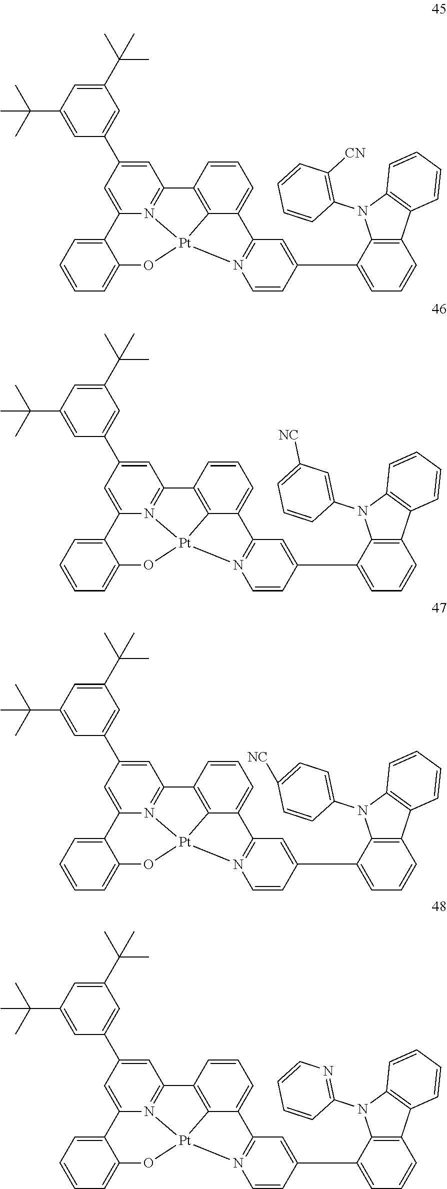

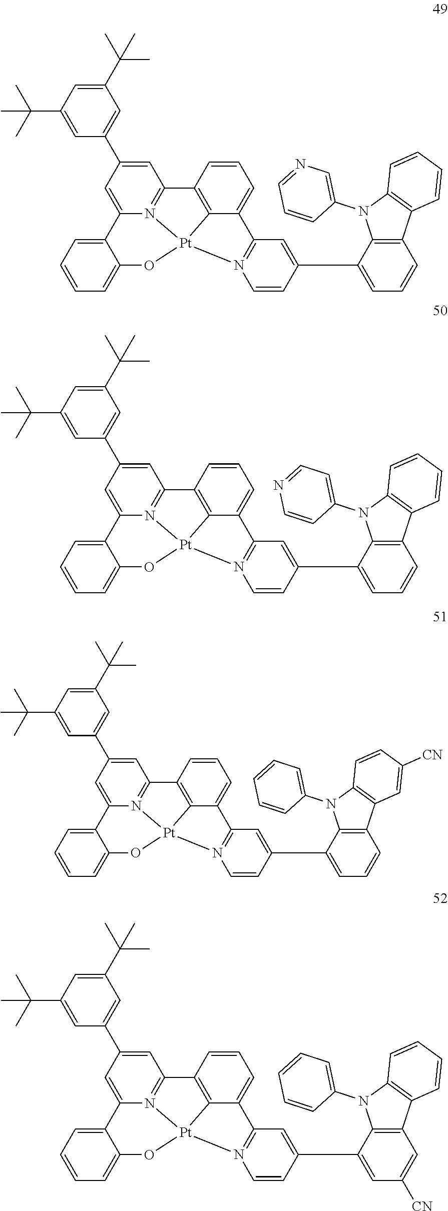

9. A precursor, that is ligand, of the platinum complex according to any one of claims 1 to 8, having the following structural formula:

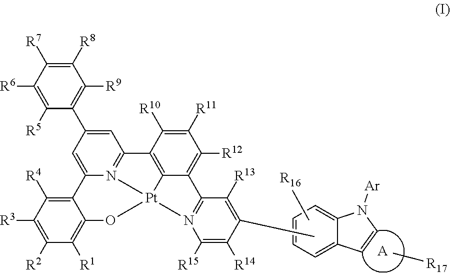

10. Application of the platinum complex according to any one of claims 1 to 8 in organic light-emitting diodes, organic thin-film transistors, organic photovoltaic devices, light-emitting electrochemical cells, or chemical sensors.

11. An organic light-emitting diode, comprising a cathode, an anode, and an organic layer, wherein the organic layer is one or more of a hole injection layer, a hole transport layer, a light-emitting layer, a hole blocking layer, an electron injection layer, and an electron transport layer, and comprises the platinum complex according to any one of claims 1 to 8.

12. The organic light-emitting diode according to claim 11, wherein a layer where the platinum complex according to any one of claims 1 to 8 is located is the light-emitting layer.
